SEQUENCE LISTING (4 sequences)

SEQ ID NO: 1 - protein, 18 aa, synthetic construct
GAGAGAGAGAGAGAGAGA

SEQ ID NO: 2 - protein, 20 aa, synthetic construct
GAGAGAGAGAGAGAGAGAGA

SEQ ID NO: 3 - DNA, 132 bp, synthetic construct
  source 1..132: mol_type = other DNA
ggggctggggctggggctggggctggggctggggctggggctggggctggggctggggctggggctggggctggggctggggctggggctggggctggggctggggctggggctggggctggggctggggct

SEQ ID NO: 4 - DNA, 18 bp, synthetic construct
  source 1..18: mol_type = other DNA
ggggctgaggctggggct

(19) United States
(12) Patent Application Publication
(10) Pub. No.: US 2024/0426844 A1
(43) Pub. Date: Dec. 26, 2024

(54) POLY-GA PROTEINS IN ALZHEIMER’S DISEASE

(71) Applicant: University of Florida Research Foundation, Incorporated, Gainesville, FL (US)

(72) Inventors: Laura Ranum, Gainesville, FL (US); Lien Nguyen, Gainesville, FL (US)

(73) Assignee: University of Florida Research Foundation, Incorporated, Gainesville, FL (US)

(21) Appl. No.: 18/706,038

(22) PCT Filed: Nov. 1, 2022
(86) PCT No.: PCT/US2022/079048

§ 371 Date: Apr. 30, 2024

Related U.S. Application Data
(60) Provisional application No. 63/274,210, filed on Nov. 1, 2021.

(51) Int. Cl. G01N 33/68; C07K 16/44; C12N 15/113; G01N 33/53; G01N 33/58

(52) U.S. Cl. CPC G01N 33/6896; C07K 16/44; C12N 15/113; G01N 33/53; G01N 33/58; C12N 2310/11; C12N 2310/14; G01N 2800/2821

(57) ABSTRACT

Aspects of the disclosure relate to compositions and methods for the diagnosis and/or treatment of certain neurodegenerative diseases, for example those diseases associated with repeat-associated non-ATG (RAN) translation proteins, such as Alzheimer's disease (AD). In some embodiments, the disclosure relates to identifying a subject having a RAN protein-associated disease by detecting expression or activity of repeat-associated non-ATG (RAN) translation proteins (e.g., RAN proteins). In some embodiments, the disclosure relates to methods of treating a RAN protein-associated disease by administering to a subject in need thereof an agent that reduces expression or activity of RAN proteins.

### RELATED APPLICATIONS

This application claims the benefit under 35 U.S.C. § 119(e) of U.S. Provisional Application No. 63/274,201, filed Nov. 1, 2021, the entire contents of which are incorporated by reference herein.

### STATEMENT OF FEDERAL FUNDING

This invention was made with government support under Grant Nos. K99 AG065511, NS126536 and R01 NS098819 awarded by the National Institutes of Health, and Grant No. W81XWH-22-1-0592 awarded by the U.S. Army Medical Acquisition Activity. The government has certain rights in the invention.

### BACKGROUND

Microsatellite repeat expansions are known to cause more than forty neurodegenerative disorders. Molecular features common to many of these disorders include the accumulation of RNA foci containing sense and antisense expansion transcripts and the accumulation of proteins from repeat-associated non-AUG (RAN) translation. RAN translation can occur across a broad range of repeat lengths from pre-mutation lengths (˜30-40 repeats) to full expansions (up to 10,000 repeats). While repetitive elements account for a large portion of the human genome, the detection of repeats and repeat expansion mutations is challenging.

### SUMMARY

Aspects of the disclosure relate to methods and compositions for identification and/or treatment of diseases associated with repeat-associated non-ATG (RAN) protein translation. The disclosure is based, in part, on the recognition that certain genetic mutations in a subject cause translation of poly-Glycine-Alanine (poly-GA) repeat-containing proteins (e.g., poly-GA RAN proteins) in subjects having certain neurodegenerative diseases, for example Alzheimer's disease (AD). As described in the Example, such poly-GA RAN proteins accumulate in tissue (e.g., neurological tissue, such as neurons, glial cells, etc.) of subjects having AD. The present inventors surprisingly discovered that this accumulation of poly-GA RAN proteins (e.g., poly-GA RAN proteins expressed from certain RNA transcripts, for example RNA transcripts set forth in Table 2) in subjects having AD is associated with earlier age of death in the subjects.

Accordingly, in some aspects, the disclosure provides a method for treating a subject having or suspected of having Alzheimer's disease (AD), the method comprising administering to the subject one or more therapeutic agents that reduce poly-(Glycine-Alanine) (poly-GA) repeat-associated non-ATG (RAN) protein translation, expression, aggregation or accumulation.

In some embodiments, the subject comprises one or more mutations at a chromosomal locus or in a gene set forth in Table 2. In some embodiments, the subject comprises one or more mutations at a chromosomal locus in an X chromosome (chrX) beginning at 100748986 and ending at 100749205. In some embodiments, the subject comprises one or more mutations in ARMCX4.

In some embodiments, the poly-GA RAN protein is translated from an mRNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNM1P46, DNM1P35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCK5, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A. In some embodiments, the poly-GA RAN protein is translated from an mRNA transcript encoded by a chromosomal locus in chrX beginning at 100748986 and ending at 100749205. In some embodiments, the poly-GA RAN protein is translated from an mRNA transcript encoded by ARMCX4.

In some embodiments, the one or more therapeutic agents comprise an anti-RAN protein antibody, an inhibitory nucleic acid, a peptide, and/or a small molecule.

In some embodiments, the anti-RAN protein antibody is an anti-poly-GA antibody. In some embodiments, the anti-poly-GA antibody specifically binds to a poly-GA repeat region of the RAN protein in the subject. In some embodiments, the anti-poly-GA antibody is a monoclonal antibody.

In some embodiments, the inhibitory nucleic acid is double-stranded RNA (dsRNA), short-interfering RNA (siRNA), short-hairpin RNA (shRNA), microRNA (miRNA), artificial microRNA (amiRNA), an aptamer, or an antisense oligonucleotide (ASO).

In some embodiments, the inhibitory nucleic acid comprises a region of complementarity with a nucleic acid sequence present at a chromosomal locus set forth in Table 2, or wherein the inhibitory nucleic acid comprises a region of complementarity with an RNA transcript encoded by a gene set forth in Table 2. In some embodiments, the inhibitory nucleic acid comprises a region of complementarity with a nucleic acid sequence encoding a poly-GA repeat expansion in the subject. In some embodiments, the inhibitory nucleic acid comprises a region of complementarity with an RNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNMIP46, DNMIP35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A. In some embodiments, the inhibitory nucleic acid comprises a region of complementarity with an RNA transcript encoded by a chromosomal locus in chrX beginning at 100748986 and ending at 100749205. In some embodiments, the inhibitory nucleic acid comprises a region of complementarity with an RNA transcript encoded by ARMCX4.

In some embodiments, the subject is a mammal. In some embodiments, the subject is a human. In some embodiments, the agent is administered to a central nervous (CNS) cell of the subject. In some embodiments, the CNS cell is a neuron or glial cell.

In some embodiments, the administration of the one or more therapeutic agents to the subject results in a reduction of poly-GA RAN protein translation, expression, aggregation, or accumulation in the subject, relative to the level of poly-GA RAN protein translation, expression, aggregation, or accumulation in the subject prior to the administration.

Aspects of the disclosure relate to a method of identifying a subject as having or likely to develop Alzheimer's disease (AD). In some embodiments, the method comprises: (i) obtaining a biological sample from the subject; (ii) contacting the biological sample with detectable agent specific for a poly-(Glycine-Alanine) (poly-GA) repeat-associated non-ATG (RAN) protein; (iii) performing an assay on the biological sample to detect the presence of poly-GA RAN proteins, wherein poly-GA RAN proteins are detected via the detectable agent; and (iv) identifying the subject as having or likely to develop AD based upon the detection of the detectable agent. In some embodiments, the method comprises: (i) obtaining a biological sample from the subject; (ii) contacting the biological sample with detectable agent specific for a poly-(Glycine-Alanine) (poly-GA) repeat-associated non-ATG (RAN) protein; (iii) performing an assay on the biological sample to detect the presence of poly-GA RAN proteins (e.g., a poly-GA RAN protein encoded by a gene of Table 2), wherein poly-GA RAN proteins are detected via the detectable agent; (iv) identifying the subject as having or likely to develop AD based upon the detection of the detectable agent; and (v) administer one or more therapeutic agents to the subject when the subject is identified as having or likely to develop AD based upon the detection of the detectable agent.

In some embodiments, the poly-GA RAN protein is encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNMIP46, DNMIP35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A. In some embodiments, the poly-GA RAN protein is translated from an mRNA transcript encoded by a chromosomal locus in chrX beginning at 100748986 and ending at 100749205. In some embodiments, the poly-GA RAN protein is translated from an mRNA transcript encoded by ARMCX4.

In some embodiments, the subject is identified as having AD based upon the detection of the detectable agent. In some embodiments, the subject is identified as likely to develop AD based upon the detection of the detectable agent.

In some embodiments, the biological sample is tissue, blood, serum, or cerebrospinal fluid (CSF), optionally wherein the tissue is brain tissue or spinal cord tissue.

In some embodiments, the assay comprises an antibody-based capture assay, binding assay, hybridization assay, immunoblot analysis, Western blot analysis, immunohistochemistry, dCas9-based enrichment, label free immunoassays, immunoquantitative PCR, mass spectrometry, bead-based immunoassays, immunoprecipitation, immunostaining, immunoelectrophoresis, and/or ELISA.

In some embodiments, the dCas9 protein is Streptococcus pyogenes dCas9 (spdCas9).

In some embodiments, the ELISA is RCA-based ELISA or rtPCR-based ELISA. In some embodiments, the detectable agent comprises an anti-poly-GA antibody.

In some embodiments, the anti-poly-GA antibody is linked to a detectable label. In some embodiments, the detectable label comprises an enzyme, prosthetic group, fluorescent material, luminescent material, bioluminescent material, radioactive material, positron emitting metal, nonradioactive paramagnetic metal ion, or affinity label.

In some embodiments, the subject is mammal. In some embodiments, the subject is a human.

In some embodiments, the subject identified as having or likely to develop AD is administered one or more therapeutic agents. In some embodiments, the one or more therapeutic agents comprises an anti-RAN protein antibody, an inhibitory nucleic acid, a peptide, and/or a small molecule.

In some embodiments, the anti-RAN protein antibody is an anti-poly-GA antibody. In some embodiments, the anti-poly-GA antibody specifically binds to a poly-GA repeat region of the RAN protein in the subject. In some embodiments, the anti-poly-GA antibody is a monoclonal antibody.

In some embodiments, the inhibitory nucleic acid is double-stranded RNA (dsRNA), short-interfering RNA (siRNA), short-hairpin RNA (shRNA), microRNA (miRNA), artificial microRNA (amiRNA), an aptamer, or an antisense oligonucleotide (ASO). In some embodiments, the inhibitory nucleic acid comprises a region of complementarity with a nucleic acid sequence encoding a poly-GA repeat expansion in the subject. In some embodiments, the inhibitory nucleic acid comprises a region of complementarity with a nucleic acid sequence present at a chromosomal locus set forth in Table 2. In some embodiments, the inhibitory nucleic acid comprises a region of complementarity with an RNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNM1P46, DNM1P35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A. In some embodiments, the inhibitory nucleic acid comprises a region of complementarity with an RNA transcript encoded by a chromosomal locus in chrX beginning at 100748986 and ending at 100749205. In some embodiments, the inhibitory nucleic acid comprises a region of complementarity with an RNA transcript encoded by ARMCX4.

In some embodiments, the administration of the one or more therapeutic agents to the subject results in a reduction of poly-GA RAN protein translation, expression, aggregation or accumulation in the subject, relative to the level of poly-GA RAN protein translation, expression, aggregation or accumulation in the subject prior to the administration.

### BRIEF DESCRIPTION OF DRAWINGS

FIG. 1 shows representative data indicating that poly-GA RAN proteins accumulate in Alzheimer's disease (AD) tissue, as measured by staining with a poly-GA repeat-specific antibody.

FIGS. 2A-2B show representative data relating to translation of poly-GA repeat proteins in AD. FIG. 2A shows data indicating that poly-GA inclusions are up-regulated in AD tissue relative to control (e.g., non-AD) tissue. FIG. 2B shows data indicating that higher poly-GA staining correlates with an earlier age of death in AD patients.

FIGS. 3A-3B show representative data relating to aggregation and accumulation patterns of poly-GA repeat proteins in AD. FIG. 3A shows data indicating that poly-GA repeat proteins co-localize with the protein LCB3 in AD patients. FIG. 3B shows control data, wherein no poly-GA proteins were observed.

FIG. 4 shows a predicted ARMCX4 poly-GA repeat protein. Alternative splicing variants are shown, demonstrating that an ARMCX4 repeat expansion can be in an intron (left) or an exon (right). The expanded allele contains 553 base pairs (bp) and contains ˜90 repeats.

FIG. 5 shows representative data relating to aggregation and accumulation patterns of poly-GA repeat proteins in AD. Data is shown indicating that poly-GA repeat proteins co-localize with the protein ARMCX4 in AD patients (top row). Neither poly-GA repeat proteins nor ARMCX4 were observed in controls (bottom row).

FIG. 6 shows representative data relating to detection of GA-rich proteins expressed from the ARMCX4 repeat expansion, using an anti-GA antibody. Data is shown indicating that GA-rich proteins expressed from the ARMCX4 repeat expansion are able to be detected using an anti-GA antibody (top row). No poly-GA repeat proteins were detected in controls (bottom row).

### DETAILED DESCRIPTION

Aspects of the disclosure relate to methods and compositions for identification and/or treatment of diseases associated with repeat-associated non-ATG (RAN) protein translation. The disclosure is based, in part, on the recognition that certain genetic mutations in a subject cause translation of poly-Glycine-Alanine (poly-GA) repeat-containing proteins in subjects having Alzheimer's disease. As described in the Example, such poly-GA proteins aggregate and accumulate in tissue (e.g., neurological tissue, such as neurons, glial cells, etc.) of subjects having Alzheimer's disease (AD). It has been observed that this aggregation and/or accumulation of poly-GA RAN proteins in subjects having AD is associated with earlier age of death in the subjects. In some embodiments, a poly-GA RAN protein is translated from a chromosomal locus or a gene set forth in Table 2.

As used herein, the terms “poly-GA repeat protein” and “poly-GA RAN protein” are used interchangeably to refer to a protein having Glycine-Alanine (GA) amino acid repeat motifs. The repeat motifs may vary in length. In some embodiments, a poly-GA RAN protein comprises between 2 and 100 “GA” repeats. In some embodiments, a poly-GA RAN protein comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 “GA” repeats. In some embodiments, a poly-GA RAN protein comprises more than 100 “GA” repeats.

Biological samples can be any specimen derived or obtained from a subject having or suspected of having a disease (e.g., neurological disease) associated with RAN protein expression, translation, aggregation and/or accumulation, for example AD. In some embodiments, the biological sample is blood, serum (e.g., plasma from which the clotting proteins have been removed) or cerebrospinal fluid (CSF). In some embodiments, a biological sample is a tissue sample, for example central nervous system (CNS) tissue, such as brain tissue or spinal cord tissue. The skilled artisan will recognize other biological samples, such as cells (e.g., brain cells, neuronal cells, skin cells, etc.) suitable for methods described by the disclosure.

A “subject having or suspected of having a disease (e.g., neurological diseases) associated with RAN protein expression, translation, aggregation and/or accumulation” generally refers to a subject exhibiting one or more signs and symptoms of a neurodegenerative disease, including but not limited to memory deficit (e.g., short term memory loss), confusion, deficiencies of executive functions (e.g., attention, planning, flexibility, abstract thinking, etc.), loss of speech, degeneration or loss of motor skills, etc., or a subject having or being identified as having one or more genetic mutations associated with RAN protein expression, translation, aggregation and/or accumulation.

A “subject having or suspected of having Alzheimer's disease (AD)” can be a subject exhibiting one or more signs and symptoms of AD, including but not limited to memory deficit (e.g., short term memory loss), confusion, deficiencies of executive functions (e.g., attention, planning, flexibility, abstract thinking, etc.), loss of speech, degeneration or loss of motor skills, etc., or a subject having or being identified as having one or more genetic mutations associated with AD, for example mutations in specific genes including apolipoprotein (APP), presenilin genes (PSEN1 and PSEN2), or MAPT (e.g., encoding a tau protein). In some embodiments, a subject having or suspected of having AD is characterized by the accumulation of 0-amyloid (A3) peptides and/or hyper-phosphorylated tau protein throughout brain tissue of the subject. In some embodiments, a subject has been diagnosed as having AD by a medical professional, according to the NINCDS-ADRDA Alzheimer's Criteria, as described by McKhann et al. (1984) “Clinical diagnosis of Alzheimer's disease: report of the NINCDS-ADRDA Work Group under the auspices of Department of Health and Human Services Task Force on Alzheimer's Disease”. Neurology. 34 (7): 939-44. A subject can be a mammal (e.g., human, mouse, rat, dog, cat, or pig). In some embodiments, a subject is a non-human animal, for example a mouse, rat, guinea pig, cat, dog, horse, camel, etc. In some embodiments, the subject is a human.

### RAN Proteins

A “RAN (repeat-associated non-ATG translated) protein” is a polypeptide that is translated from sense or antisense RNA sequences bidirectionally transcribed from a repeat expansion mutation in the absence of an AUG initiation codon. In some embodiments, the RAN protein is a poly-Glycine-Alanine (poly-GA) repeat-containing RAN protein. Poly-GA RAN protein-encoding sequences can be found in the genome (e.g., human genome) at multiple loci, including but not limited to the loci and sequences set forth in Table 2. In some embodiments, a subject is characterized as having a mutation in one or more chromosomal loci or genes set forth in Table 2, where the one or more mutations results in translation of one or more poly-GA RAN proteins. In some embodiments, the one or more mutations result in the locus or gene having an abnormal repeat expansion, for example the repeat expansions described in Table 1.

Generally, poly-GA RAN proteins are translated from abnormal repeat expansions (e.g., as shown in Table 1) of DNA. In some embodiments, the disease status of a subject having or suspected of having a RAN protein-associated disease is classified by the number and/or type of microsatellite repeats present (e.g., detected) in the subject (e.g., in the genome of a subject or in a gene of the subject). In some embodiments, a subject having less than 10 repeat sequences does not exhibit signs or symptoms of a RAN protein-associated disease characterized by RAN protein translation. In some embodiments, a subject having between 10 and 40 repeats may or may not exhibit one or more signs or symptoms of a RAN protein-associated disease characterized by RAN protein translation. In some embodiments, a subject having more than 40 repeats exhibits one or more signs or symptoms of a RAN protein-associated disease characterized by RAN protein translation. In certain cases, a subject is identified as having a RAN protein-associated disease characterized by a large (e.g., >100) number of repeats. Microsatellite repeat sequences encoding RAN proteins are generally known. In some embodiments, the RAN protein-associated disease is Alzheimer's disease (AD).

### Methods of Detecting RAN Proteins

The disclosure is based, in part, on the discovery that certain biological sample processing methods (e.g., antibody-based capture, hybridization-based assays, dCas9-based enrichment, or combinations thereof) enable the reproducible detection of one or more RAN proteins in a biological sample. In some embodiments of methods described by the disclosure, a sample (e.g., a biological sample) is treated by an antibody-based capture process to isolate one or more RAN proteins within the sample. Typically, the antibody-based capture methods include contacting the sample with one or more (e.g., 2, 3, 4, 5, or more) anti-RAN protein antibodies. In some embodiments, the one or more anti-RAN antibodies are conjugated to a solid support (e.g., a scaffold, resin beads, etc.). In some embodiments, antibody-based capture methods comprise physically separating and/or isolating RAN proteins that have been bound by the anti-RAN antibody(s), for example eluting the RAN proteins by a chromatographic method such as affinity chromatography or ion-exchange chromatography.

A biological sample may be subjected to an antigen retrieval procedure prior to being contacted with an anti-RAN antibody. As used herein, “antigen retrieval” (also referred to as epitope retrieval, or antigen unmasking) refers to a process in which a biological sample (e.g., blood, serum, CSF, etc.) is treated under conditions which expose antigens (e.g., epitopes) that were previously inaccessible to detection agents (e.g., antibodies, aptamers, and other binding molecules) prior to the process. Generally, antigen retrieval methods comprise steps including but not limited to heating, pressure treatment, enzymatic digestion, treatment with reducing agents, treatment with oxidizing agents, treatment with crosslinking agents, treatment with denaturing agents (e.g., detergents, ethanol, acids), or changes in pH, or any combination of the foregoing. Several antigen retrieval methods are known in the art, including but not limited to protease-induced epitope retrieval (PIER) and heat-induced epitope retrieval (HIER). In some embodiments, antigen retrieval procedures reduce the background and increase the sensitivity of detection techniques (e.g., immunohistochemistry (IHC), immuno-blot (such as Western Blot), ELISA, etc.).

Detection of RAN proteins in a biological sample may be performed by Western blot. Western blots generally employ the use of a detection agent or probe to identify the presence of a protein or peptide. In some embodiments, detection of one or more RAN proteins is performed by immunoblot (e.g., dot blot, 2-D gel electrophoresis, Western Blot, etc.), immunohistochemistry (IHC), ELISA (e.g., RCA-based ELISA or rtPCR-based ELISA), label free immunoassays such as surface plasmon resonance bio layer interferometry, immunoquantitative PCR, mass spectrometry such as GC-MS, LC-MS, MALDI-TOF-MS, bead-based immunoassays, immunoprecipitation, immunostaining, or immunoelectrophoresis. In some embodiments, the detection agent is an antibody. In some embodiments, the antibody is an anti-RAN protein antibody, such as an anti-poly(GA) antibody. In some embodiments, an anti-RAN protein antibody targets (e.g., specifically binds to) the amino acid repeat region (e.g., GAGAGAGAGAGAGAGAGA (SEQ ID NO: 1), etc.) of a RAN protein. In some embodiments, an anti-RAN protein antibody targets (e.g., specifically binds to) an epitope comprising amino acids in the characteristic reading frame-specific C-terminus translated 3′ of the repeated amino acids. In some embodiments, an anti-RAN protein antibody targets (e.g., specifically binds to) an epitope comprising amino acids bridging the C-terminus of the amino acid repeat region and the N-terminus of the characteristic reading-frame specific C-terminus translated 3′ of the repeated amino acids.

In some embodiments, an anti-RAN antibody targets (e.g., specifically binds to) any portion of a RAN protein that does not comprise the poly-amino acid repeat, for example the C-terminus of a RAN protein (e.g., the C-terminus of a poly(GA) repeat protein). Examples of anti-RAN antibodies targeting RAN protein poly-amino acid repeats are disclosed, for example, in International Application Publication No. WO 2014/159247, the entire content of which is incorporated herein by reference. Examples of anti-RAN antibodies targeting the C-terminus of RAN proteins are disclosed, for example, in U.S. Publication No. 2013/0115603, the entire content of which is incorporated herein by reference.

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum). In some embodiments, the antigen is 12-20 amino acids. For antibodies against repeat motifs, an antigen is a repeat sequence. For antibodies against C-terminal sequence of a RAN protein, an antigen is a C-terminal-specific sequence. In some embodiments, an antigen is a portion of a C-terminal sequence, for example, a fragment of the C-terminal sequences that is 3-5 or 5-10, or more amino acids in length, for example 6, 7, 8, 9, 10, 11, 12, 13, 14, 15, 16, 17, 18, 19, 20, 25, 30, 35, 40, or 50 amino acids in length.

In some embodiments, the disclosure provides methods of producing an antibody, the method comprising administering to the subject a peptide antigen comprising a RAN protein repeat sequence, for example a poly(GA) repeat sequence. In some embodiments, the subject is a mammal, for example a non-human primate, rodent (e.g., rat, hamster, guinea pig, etc.). In some embodiments, the subject is a human (e.g., a human subject is injected with a peptide antigen for the purposes of eliciting a host antibody response against the peptide antigen, for example a RAN protein). In some embodiments, an antibody is produced by expressing in a cell (e.g., a B-cell, hybridoma cell, etc.) one or more RAN proteins or RAN protein repeat sequences.

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256: 495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809.

In addition to the use of display libraries, the specified antigen (e.g., one or more RAN proteins) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825).

For additional antibody production techniques, see, Antibodies: A Laboratory Manual, Second Edition. Edited by Edward A. Greenfield, Dana-Farber Cancer Institute, ©2014. The present disclosure is not necessarily limited to any particular source, method of production, or other special characteristics of an antibody.

In some embodiments, methods of detecting one or more RAN proteins (e.g., poly(GA) RAN proteins) in a biological sample are useful for monitoring the progress of a disease associated with RAN protein expression, translation, aggregation and/or accumulation (e.g., AD). In some embodiments, the disease associated with RAN protein expression, translation, aggregation and/or accumulation is Alzheimer's Disease (AD). Aspects of the disclosure thus relate to a method for monitoring a therapeutic treatment course for a disease associated with RAN protein (e.g., poly(GA) RAN protein) translation (e.g., AD). In some embodiments, the method comprises: (i) performing an assay on a first biological sample obtained from a subject, (ii) performing an assay on a second biological sample obtained from a subject, (iii) detecting the presence or absence of RAN proteins (e.g., poly(GA) RAN proteins) in the first and second biological samples, and (iv) comparing the level or quantity of RAN proteins present in the second blood sample to the level or quantity of RAN proteins in the first blood sample. In some embodiments, the assay comprises a step of contacting the biological sample with one or more anti-RAN protein antibodies (e.g., anti-poly(GA) antibodies). In some embodiments, the first biological sample is obtained before administration of a therapeutic agent to the subject. In some embodiments, the second biological sample is obtained after administration of a therapeutic agent to the subject.

For example, in some embodiments, biological samples are obtained from a subject prior to and after (e.g., 1 week, 2 weeks, 1 month, 6 months, or one year after) commencement of a therapeutic regimen (e.g., an AD therapeutic regimen) and the amount of RAN proteins (e.g., poly(GA) RAN proteins) detected in the samples is compared. In some embodiments, if the level (e.g., amount) of RAN protein (e.g., poly(GA) RAN protein) in the post-treatment sample is reduced compared to the pre-treatment level (e.g., amount) of RAN protein (e.g., poly(GA) RAN protein), the therapeutic regimen is successful. In some embodiments, if the level (e.g., amount) of RAN protein in the post-treatment sample is unaltered compared to the pre-treatment level (e.g., amount) of RAN protein, the therapeutic regimen is successful (e.g., the therapeutic regimen was successful in halting or slowing disease progression). In some embodiments, if the level (e.g., amount) of RAN protein in the post-treatment sample is unaltered compared to the pre-treatment level (e.g., amount) of RAN protein, the therapeutic regimen is not successful (e.g., the therapeutic regimen was not successful in reducing RAN protein levels). In some embodiments, if the level (e.g., amount) of RAN protein in the post-treatment sample is increased compared to the pre-treatment level (e.g., amount) of RAN protein, the therapeutic regimen is not successful. In some embodiments, the level of RAN proteins (e.g., poly(GA) RAN proteins) in biological samples (e.g., 1, 2, 3, 4, 5, 6, 7, 8, 9, 10, or more samples) of a subject are continuously monitored during a therapeutic regimen (e.g., an AD therapeutic regimen) (e.g., measured on 1, 2, 3, 4, 5, 6, 7, 8, 9, 10, or more separate occasions).

In some embodiments, methods described by the disclosure comprise a step of administering a therapeutic agent (e.g., one or more therapeutic agents for the treatment of AD) to the subject if the level of RAN proteins (e.g., poly(GA) RAN proteins) detected in the first biological sample is elevated compared to a level of RAN proteins (e.g., poly(GA) RAN proteins) detected in a control sample. In some embodiments, methods described by the disclosure comprise a step of continuing to administer a therapeutic agent (e.g., one or more therapeutic agents for the treatment of AD) to the subject if the level of RAN proteins (e.g., poly(GA) RAN proteins) detected in the second biological sample is reduced or unchanged compared to a level of RAN proteins (e.g., poly(GA) RAN proteins) detected in the first biological sample. In some embodiments, methods described by the disclosure comprise a step of stopping administration of a therapeutic agent (e.g., one or more therapeutic agents for the treatment of AD) to the subject if the level of RAN proteins (e.g., poly(GA) RAN proteins) detected in the second biological sample is elevated or unchanged compared to a level of RAN proteins (e.g., poly(GA) RAN proteins) detected in the first biological sample.

Controls and control levels include RAN protein levels obtained (e.g., detected) from a subject that does not have or is not suspected of having a disease or disorder associated with RAN protein translation, for example AD. In some embodiments, a control sample refers to a sample obtained from a subject having or suspected of having a disease associated with RAN protein translation (e.g., AD) prior to the administration of a therapeutic agent to the subject.

In some embodiments, a detection agent is an aptamer (e.g., RNA aptamer, DNA aptamer, or peptide aptamer). In some embodiments, an aptamer specifically binds to a RAN protein (e.g., poly(GA) protein). In some embodiments, an aptamer specifically binds to a nucleotide sequence encoding a RAN protein (e.g., a nucleotide sequence encoding a poly(GA) protein).

Aspects of the disclosure relate to nucleic acid hybridization-based methods for identifying the presence of RAN proteins or microsatellite repeat sequences encoding RAN proteins in a biological sample (e.g., a biological sample obtained from a subject). The disclosure is based, in part, on methods for detecting nucleic acid sequences encoding RAN proteins by detectable nucleic acid probes (e.g., fluorophore-conjugated DNA probes). Generally, a “detectable nucleic acid probe” refers to a nucleic acid sequence that specifically binds to (e.g., hybridizes with) a target sequence, and comprises a detectable moiety, for example a fluorescent moiety, radioactive moiety, chemiluminescent moiety, electroluminescent moiety, biotin, peptide tag (e.g., poly-His tag, FLAG-tag, etc.), etc. In some embodiments, the detectable nucleic acid probe comprises a region of complementarity (e.g., a nucleic acid sequence that is the complement of, and capable of hybridizing to) a nucleic acid sequence encoding one or more RAN proteins. A region of complementarity may range from about 2 nucleotides in length to about 100 nucleotides in length (e.g., any number of nucleotides between 2 and 100, inclusive). In some embodiments, A region of complementarity may range from about 2 nucleotides in length to about 50 nucleotides in length, from about 2 nucleotides in length to about 25 nucleotides in length, from about 2 nucleotides in length to about 18 nucleotides in length, from about 2 nucleotides in length to about 15 nucleotides in length, from about 2 nucleotides in length to about 10 nucleotides in length, or from about 2 nucleotides in length to about 5 nucleotides in length.

In some embodiments, a nucleic acid probe comprises a region of complementarity with a genomic locus or gene set forth in Table 2, or a region of complementarity with a repeat sequence comprising multiple repeats of a sequence set forth in Table 1. In some embodiments, a detectable nucleic acid probe is a DNA probe. In some embodiments, the DNA probe is conjugated to a fluorophore.

A biological sample may also be contacted with a plurality of detectable nucleic acid probes. The number of nucleic acid probes in a plurality varies. In some embodiments, a plurality of nucleic acid probes comprises between 2 and 100 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100) nucleic acid probes. In some embodiments, a plurality comprises more than 100 probes. The nucleic acid probes may be the same or different sequences. In some embodiments, a plurality of detectable nucleic acid probes comprises probes which hybridize to nucleic acid sequences that encode a poly(GA) RAN protein (e.g., repeat sequences set forth in Table 1).

In some embodiments, detectable nucleic acid probes are useful for localization of RAN protein translation by Fluorescence In situ Hybridization (FISH).

Methods for detecting one or more RAN proteins may comprise an enrichment step. “Enrichment” refers to processes which increase the amount and/or concentration of a target nucleic acid in a sample relative to other nucleic acids in a sample. Generally, enrichment may occur by increasing the number of target nucleic acid sequences in a sample (e.g., by amplifying the target sequence, for example by polymerase chain reaction (PCR), etc.), or by decreasing the amount or concentration of non-target nucleic acid sequences in the sample (e.g., by separating or isolating the target nucleic acid sequence from non-target sequences).

In some embodiments, methods described herein comprise a step of enriching a biological sample for nucleic acid sequences (e.g., microsatellite repeat sequences) encoding RAN proteins. In some embodiments, the enrichment comprises contacting the biological sample with (1) a labeled (e.g., biotinylated) dCas9 protein, and (2) one or more single-stranded guide RNA (sgRNAs) that specifically bind to nucleic acid repeat sequences encoding RAN proteins. In some embodiments, the labeled dCas9 protein and the one or more sgRNAs are provided together as a single molecule (e.g., a dCas9-sgRNA complex). In some embodiments after the biological sample with the labeled dCas9 protein and the one or more sgRNAs, the nucleic acid sequences encoding one or more RAN proteins are isolated from the labeled dCas9 protein and the sgRNAs, for example by affinity chromatography, as described by Liu et al. (2017) Cell 170: 1028-1043.

In some embodiments, the detection of the one or more RAN proteins comprises Next-Generation Sequencing (NGS). In some embodiments, an enrichment step (e.g., dCas9-based enrichment) is performed on the sample, using guideRNAs. In some embodiments, the guideRNAs used in the enrichment target NGG protospacer adjacent motifs (PAM) containing repeats. In other embodiments, the guideRNAs used in the enrichment target non-NGG PAM containing repeats. In some embodiments, the non-NGG PAM containing repeats comprise one or more of the expansion repeat sequences set forth in Table 1. In some embodiments, the guideRNAs used in the enrichment enrich non-NGG PAM containing repeat expansions that are longer (e.g., 1, 2, 3, 4, 5, 6, 7, 8, 9, 10, 11, 12, 13, 14, 15, 20, 25, 30, 35, 40, 45, 50, 55, 60, 70, 80, 90, or 100 repeats longer, or more) than the corresponding normal allele. In some embodiments, the guideRNAs used in the enrichment identify multiple repeat expansions simultaneously, including, in some embodiments, sequences with non-NGG PAMs.

### Therapeutic Methods

Methods of treating a disease associated with RAN protein expression, translation, and/or accumulation are also contemplated by the disclosure. In some embodiments, the disease associated with RAN proteins is Alzheimer's Disease (AD). In some embodiments, a subject having been diagnosed with a disease associated with RAN proteins by a method described by the disclosure is administered a therapeutic useful for treating a disease associated with RAN proteins.

To “treat” a disease (e.g., AD) as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host cell, tissue or organ. A therapeutically acceptable amount of an anti-RAN protein antibody may be an amount that is capable of treating a disease, e.g., Alzheimer's disease, by reducing expression, accumulation and/or aggregation of RAN proteins and/or appearance or number of RNA foci comprising RAN protein-encoding microsatellite repeat sequences. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

A therapeutic useful for treating a disease associated with RAN proteins can be, without limitation, a small molecule, protein (e.g., antibody), peptide, nucleic acid (e.g., an interfering nucleic acid), or gene therapy vector (e.g., viral vector encoding a therapeutic protein and/or an interfering nucleic acid). Therapeutics useful for treating a disease associated with RAN proteins may target (e.g., reduce expression, activity, accumulation, aggregation, etc.) of a RAN protein or nucleic acid encoding a RAN protein, and/or modulate the activity of another gene or gene product (e.g., protein) that interacts with one or more RAN proteins. Examples of genes and gene products that interact with one or more RAN proteins include eukaryotic initiation factor 2 (eIF2), eukaryotic initiation factor 3 (eIF3), protein kinase R (PKR), p62, LC3 I subunit, LC3 II subunit, and Toll-like receptor 3 (TLR3). In some embodiments, a therapeutic agent inhibits expression or activity of one or more of: eukaryotic initiation factor 2 (eIF2), eukaryotic initiation factor 3 (eIF3), protein kinase R (PKR), p62, LC3 I subunit, LC3 II subunit, and Toll-like receptor 3 (TLR3).

In some embodiments, the therapeutic agent is a small molecule. In some embodiments, the small molecule inhibits expression or activity of one or more RAN proteins. In some embodiments, a small molecule is an inhibitor of eIF3 (or an eIF3 subunit). Examples of small molecule inhibitors of eIF3 include but are not limited to mTOR inhibitors (e.g., rapamycin, PP242), S6 kinase (S6K) inhibitors, etc.

In some embodiments, the small molecule inhibits expression or activity of eukaryotic initiation factor 2A (eIF2A) or eIF2α. Examples of small molecule inhibitors of eIF2A include but are not limited to salubrinal, Sal003, ISRIB, etc. In some embodiments, the small molecule in an inhibitor of TARBP2. Examples of TARBP2 inhibitors include anti-TARBP2 antibodies, interfering RNAs (e.g., dsRNA, siRNA, shRNA, miRNA, etc.) that target TARBP2, peptide inhibitors of TARBP2, and small molecule inhibitors of TARBP2. In some embodiments, the small molecule is metformin, also known as N,N-dimethylbiguanide (IUPAC N,N-Dimethylimidodicarbonimidic diamide and CAS 657-24-9), or an alternate bioactive biguanide including chloroguanide [1-[amino-(4-chloroanilino)methylidene]-2-propan-2-yl-guanidine, CAS 500-92-51, Chlorproguanil [1-[Amino-(3,4-dichloroanilino)methylidene]-2-propan-2-ylguanidine, CAS 537-21-31, buformin [N-Butylimidodicarbonimidic diamide, CAS 692-13-7] or Phenformin [2-(N-phenethylcarbamimidoyl) guanidine, CAS 114-86-3] or a pharmaceutically acceptable salt, co-crystal, tautomer, stereoisomer, solvate, hydrate, polymorph, isotopically enriched derivative, or prodrug of any of the biguanides.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. Metformin may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred. In some embodiments, the small molecule is buformin, or phenformin.

The therapeutic agent may be an anti-RAN protein antibody. In some embodiments, the anti-RAN protein antibody is an anti-poly(GA) antibody. An anti-RAN protein antibody may bind to an extracellular RAN protein, an intracellular RAN protein, or both extracellular and intracellular RAN proteins.

In some embodiments, an anti-RAN protein antibody targets (e.g., specifically binds to) an amino acid repeat region (e.g., GAGAGAGAGAGAGAGAGAGA (SEQ ID NO: 2), etc.) of a RAN protein. Examples of anti-RAN antibodies targeting RAN protein poly-amino acid repeats are disclosed, for example, in International Application Publication No. WO 2014/159247, the entire content of which is incorporated herein by reference. In some embodiments, an anti-RAN antibody targets (e.g., specifically binds to) any portion of a RAN protein that does not comprise the poly-amino acid repeat, for example the C-terminus of a RAN protein (e.g., the C-terminus of a poly(GA) protein). Examples of anti-RAN antibodies targeting the C-terminus of RAN proteins are disclosed, for example, in U.S. Publication No. 2013/0115603, the entire content of which is incorporated herein by reference. In some embodiments, a set (or combination) of anti-RAN antibodies (e.g., a combination of two or more anti-RAN antibodies) are administered to a subject for the purpose of treating a disease associated with RAN proteins (e.g., AD).

An anti-RAN antibody can be a polyclonal antibody or a monoclonal antibody. Typically, polyclonal antibodies are produced by inoculation of a suitable mammal, such as a mouse, rabbit or goat. Larger mammals are often preferred as the amount of serum that can be collected is greater. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum).

A therapeutic molecule may be an antisense oligonucleotide (ASO). In general, antisense oligonucleotides block the translation of a target protein by hybridizing to an mRNA sequence encoding the target protein, thereby inhibiting protein synthesis by ribosomal machinery. In some embodiments, the antisense oligonucleotide (ASO) targets a gene comprising a microsatellite repeat sequence. In some embodiments, the antisense oligonucleotide inhibits translation of one or more RAN proteins. One skilled in the art would understand how to construct an antisense oligonucleotide comprising a short (approximately 15 to 30 nucleotides) oligonucleotide with a base sequence complementary to the RAN mRNA. One skilled in the art will understand that complementarity to the RAN mRNA can be established using canonical nucleotides comprising ribose, phosphate and one of the bases adenine, guanine, cytosine, and uracil linked with the phosphodiester linkages typifying naturally occurring nucleic acids, OR some of the nucleotides could be modified by replacing the ribose with an alternate saccharide moiety such as 2′-deoxyribose, or 2′-O-(2-methoxyethyl) ribose, AND/OR some or all of the nucleotides could be modified by methylation, AND/OR some or all of the phosphodiester bonds between the nucleotides could be replaced with phosphorothioate linkages. Those skilled in the art will understand that modifications of several nucleotides at both the 3′ and 5′ ends of the antisense oligonucleotide to inhibit degradation by ubiquitous, terminally active RNA nucleases will improve the stability and thus half-life of the antisense oligo. However, those skilled in the art will appreciate that it is also desirable that at least some part of the antisense oligo will, once complexed with the RAN mRNA, promote the activity of Ribonuclease H to promote the enzymatic degradation of the RAN mRNA once it is complexed with the antisense oligo.

In some embodiments, the therapeutic agent is an inhibitory nucleic acid. In some embodiments, the inhibitory nucleic acid is an interfering RNA selected from the group consisting of dsRNA, siRNA, shRNA, miRNA, and ami-RNA. In some embodiments, the inhibitory nucleic acid is a nucleic acid aptamer (e.g., an RNA aptamer or DNA aptamer). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, a therapeutic agent is an effective amount of a eukaryotic initiation factor 2 (eIF2) inhibiting agent or a Protein Kinase R (PKR) inhibiting agent (e.g., an inhibitor of eIF2 and/or PKR). In some embodiments, an inhibitor of eIF2 is an inhibitor of a serine/threonine kinase. Examples of serine/threonine kinases include but are not limited to protein kinase A (PKA), protein kinase C (PKC), Mos/Raf kinases, mitogen-activated protein kinases (MAPKs), protein kinase B (AKT kinase), etc. In some embodiments, an eIF2 inhibitor is a protein kinase R (PKR) inhibitor. Inhibitors of eIF2 and PKR are described, for example in International Application Publication No. WO 2018/195110, the entire content of which is incorporated herein by reference.

In some embodiments, the therapeutic agent is a protein kinase R (PKR) variant that functions in a dominant negative manner to inhibit phosphorylation of eIF2α. As used herein, “protein kinase R (PKR) variant” refers to a protein comprising an amino acid sequence that is at least 70%, at least 80%, at least 90%, at least 95%, at least 96%, at least 97%, at least 98%, or at least 99% identical to a wild-type protein kinase R (PKR) (e.g., GenBank Accession No. NP_002750.1), wherein the variant protein comprises at least one amino acid variation (also referred to sometimes as “mutation”) relative to the amino acid sequence of the wild-type PKR.

In some embodiments, the amino acid sequence of a PKR variant is at least 75%, at least 85%, at least 95%, at least 96%, at least 97%, at least 98%, at least 99%, at least 99.5%, at least 99.6%, at least 99.7%, at least 99.8%, or at least 99.9% identical to the amino acid sequence of wild-type PKR. In some embodiments, the amino acid sequence is about 95-99.9% identical to the amino acid sequence of wild-type PKR. In some embodiments, the amino acid sequence of a PKR variant comprises at least 1, at least 2, at least 3, at least 4, at least 5, at least 6, at least 7, at least 8, at least 9, at least 10, at least 11, at least 12, at least 13, at least 14, or at least 15 different amino acid sequence variations as compared to the sequence of amino acids set forth in the amino acid sequence of wild-type PKR. In some embodiments, a PKR variant comprises a mutation at position 296 (e.g., position 296 of a human wild-type PKR). In some embodiments, the mutation at position 296 is K296R.

An eIF2 inhibitor may be a direct inhibitor or an indirect inhibitor. Generally, a direct modulator functions by interacting with (e.g., interacting with or binding to) a gene encoding eIF2 (or eIF2α), or an eIF2 protein complex. Generally, an indirect modulator functions by interacting with a gene or protein that regulates the expression or activity of eIF2 or an eIF2α (e.g., does not directly interact with a gene or protein encoding eIF2 or an eiF2α).

In some embodiments, an inhibitor eIF2 or PKR is a selective inhibitor. A “selective inhibitor” refers to an inhibitor of eIF2 or PKR that preferentially inhibits activity or expression of one type of eIF2 subunit compared with other types of eIF2 subunits, or inhibits activity or expression of PKR preferentially compared to other kinases. In some embodiments, an inhibitor of eIF2 is a selective inhibitor of eIF2α. In some embodiments, an inhibitor of eIF2 is a selective inhibitor of eIF2A. In some embodiments, an inhibitor of eIF2 is a selective inhibitor of protein kinase R (PKR), such as a selective PKR inhibitor.

Examples of proteins that inhibit eIF2 (e.g., an eIF2 subunit) include but are not limited to polyclonal anti-eIF2 antibodies, monoclonal anti-eIF2 antibodies, etc. Examples of nucleic acid molecules that inhibit eIF2 (e.g., an eIF2 subunit) include but are not limited to dsRNA, siRNA, miRNA, etc. that target a gene encoding an eIF2 subunit (e.g., a gene encoding the mRNA set forth in GenBank Accession No. NM_004094.4). Examples of small molecule inhibitors of eIF2 include but are not limited to LY 364947, eIF-2α Inhibitor II Sal003, etc.

Examples of proteins that inhibit PKR include but are not limited to certain dominant negative PKR variants (e.g., K296R PKR mutant), TARBP2, etc. Examples of nucleic acid molecules that inhibit PKR include but are not limited to dsRNA, siRNA, miRNA, etc. that target a gene encoding a PKR. Examples of small molecule inhibitors of PKR include but are not limited to 6-amino-3-methyl-2-oxo-N-phenyl-2,3-dihydro-1H-benzo[d]imidazole-1-carboxamide, N-[2-(1H-indol-3-yl)ethyl]-4-(2-methyl-1H-indol-3-yl)pyrimidin-2-amine, metformin, buformin, phenformin, etc.

Examples of nucleic acid molecules that inhibit eIF2A include but are not limited to dsRNA, siRNA, miRNA, etc. that target a gene encoding a eIF2A (e.g., a gene encoding the mRNA set forth in GenBank Accession No. NM_032025.4). Examples of small molecule inhibitors of eIF2A include but are not limited to salubrinal, Sal003, ISRIB, etc.

In some embodiments, the eIF2 inhibitor or PKR inhibitor is an interfering (e.g., inhibitory) nucleic acid. In some embodiments, the inhibitory nucleic acid is an interfering RNA selected from the group consisting of dsRNA, siRNA, shRNA, mi-RNA, and ami-RNA.

In some embodiments, the inhibitory nucleic acid is an antisense nucleic acid (e.g., an antisense oligonucleotide (ASO) or a nucleic acid aptamer (e.g., an RNA aptamer)). Generally, an inhibitory RNA molecule can be unmodified or modified. In some embodiments, an inhibitory RNA molecule comprises one or more modified oligonucleotides, e.g., phosphorothioate-, 2′-O-methyl-, etc.-modified oligonucleotides, as such modifications have been recognized in the art as improving the stability of oligonucleotides in vivo.

In some embodiments, the interfering RNA comprises a sequence that is complementary with between 5 and 50 continuous nucleotides (e.g., 5, 6, 7, 8, 9, 10, 11, 12,13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF2 subunit or a nucleic acid sequence (such as an RNA sequence) encoding PKR.

In some embodiments, a therapeutic agent is an inhibitor of Eukaryotic initiation factor 3 (eIF3), which is a multiprotein complex that is involved with the initiation phase of eukaryotic protein translation. Generally, in humans eIF3 comprises 13 non-identical subunits (e.g., eIF3a-m). Mammalian eIF3, the largest, most complex initiation factor, comprises up to 13 non-identical subunits. Typically, eIF3f is involved in many steps of translation initiation including stabilization of the ternary complex, mediating binding of mRNA to 40S subunit and facilitating dissociation of 40S and 60S ribosomal subunits. In some embodiments, therapeutic agents that inhibit expression or activity of an eIF3 subunit (e.g., eIF3f, eIF3m, eIF3h, or other eIF3 subunit) can be used to reduce or inhibit RAN translation in a cell or in a subject (e.g., a subject having Alzheimer's disease characterized by RAN protein translation). Inhibitors of eIF3 subunits are further described, for example, in International Application Publication No. WO 2017/176813, the entire content of which is incorporated herein by reference.

An eIF3 inhibitor may be a direct inhibitor or an indirect inhibitor. Generally, a direct modulator functions by interacting with (e.g., interacting with or binding to) a gene encoding eIF3, or an eIF3 protein complex, or an eIF3 subunit. Generally, an indirect modulator functions by interacting with a gene or protein that regulates the expression or activity of eIF3 or an eIF3 subunit (e.g., does not directly interact with a gene or protein encoding eIF3 or an eiF3 subunit). In some embodiments, an inhibitor of eIF3 is a selective inhibitor. A “selective inhibitor” refers to a modulator of eIF3 that preferentially inhibits activity or expression of one type of eIF3 subunit compared with other types of eIF3 subunits. In some embodiments, an inhibitor of eIF3 is a selective inhibitor of eIF3f.

An eIF3 inhibitor can be a protein (e.g., antibody), nucleic acid, or small molecule. Examples of proteins that inhibit eiF3 (e.g., an eIF3 subunit) include but are not limited to polyclonal anti-eIF3 antibodies, monoclonal anti-eIF3 antibodies, Measles Virus N protein, Viral stress-inducible protein p56, etc. Examples of nucleic acid molecules that inhibit eiF3 (e.g., an eIF3 subunit) include but are not limited to dsRNA, siRNA, miRNA, amiRNA, etc. that target a gene encoding an eIF3 subunit. Examples of small molecule inhibitors of eIF3 include but are not limited to mTOR inhibitors (e.g., rapamycin, PP242), S6 kinase (S6K) inhibitors, etc.

In some embodiments, an interfering RNA comprises a sequence that is complementary with between 5 and 50 continuous nucleotides (e.g., 5, 6, 7, 8, 9, 10, 11, 12, 13, 14, 15, 16, 17, 18, 19, 20, 21, 22, 23, 24, 25, about 30, about 35, about 40, or about 50 continuous nucleotides) of a nucleic acid sequence (such as an RNA sequence) encoding an eIF3 subunit. Examples of nucleic acid sequences encoding eIF3 subunits include GenBank Accession No. NM_003750.2 (eIF3a), GenBank Accession No. NM_003751.3 (eIF3b), GenBank Accession No. NM_003752.4 (eIF3c), GenBank Accession No. NM_003753.3 (eIF3d), GenBank Accession No. NM_001568.2 (eIF3e), GenBank Accession No. NM_003754.2 (eIF3f), GenBank Accession No. NM_003755.4 (eIF3g), GenBank Accession No. NM_003756.2 (eIF3h), GenBank Accession No. NM_003757.3 (eIF3i), GenBank Accession No. NM_003758.3 (eIF3j), GenBank Accession No. NM_013234.3 (eIF3k), GenBank Accession No. NM_016091.3 (eIF31), GenBank Accession No. NM_006360.5 (eiF3m), etc. In some embodiments, the interfering RNA is a siRNA. In some embodiments, an eIF3f siRNA is administered (e.g., Dharmacon Cat #J-019535-08). In some embodiments, an eIF3m siRNA is administered (e.g., Dharmacon Cat #J-016219-12). In some embodiments, an eIF3h siRNA is administered (e.g., Dharmacon Cat #J-003883-07).

In some embodiments, eIF3f is a negative regulator of RAN translation and decreased levels of human eIF3f are associated with decreased accumulation of RAN proteins in cells. In some embodiments, RAN translation (e.g., in cells expressing a RAN protein) is sensitive to eIF3f knockdown unlike translation from close cognate or AUG translation. In some embodiments, the translational machinery used for RAN translation is distinct from AUG and near AUG translation machinery in a cell.

In some embodiments, a therapeutic agent is an inhibitor of TLR3. An inhibitor of TLR3 can be a protein (e.g., antibody), nucleic acid, or small molecule. Examples of proteins that inhibit TLR3 include but are not limited to polyclonal anti-TLR3 antibodies, monoclonal anti-TLR3 antibodies, etc. Examples of nucleic acid molecules that inhibit TLR3 include but are not limited to dsRNA, siRNA, miRNA, amiRNA, etc. that target a gene encoding TLR3. Examples of small molecule inhibitors of TLR3 are described, for example in Cheng et al. (2011) J Am Chem Soc 133(11):3764-7.

In some embodiments, a therapeutic agent is an inhibitor of p62 protease. An inhibitor of p62 can be a protein (e.g., antibody), nucleic acid, or small molecule. Examples of proteins that inhibit p62 include but are not limited to polyclonal anti-p62 antibodies, monoclonal anti-p62 antibodies, etc. Examples of nucleic acid molecules that inhibit p62 include but are not limited to dsRNA, siRNA, miRNA, amiRNA, etc. that target a gene encoding p62. In some embodiments, a therapeutic agent is an agent that increases proteasome activity, for example as described in Leestemaker et al. (2017) Cell Chemical Biology 24, 725-736.

In some embodiments, a therapeutic agent comprises a peptide antigen that targets one or more RAN proteins (e.g., is a RAN protein vaccine that targets one or more RAN proteins). In some embodiments, the peptide antigen targets (e.g., comprises an amino acid sequence encoding) a poly(Glycine-Alanine) [poly(GA)] repeat-containing protein.

In some embodiments, one or more therapeutic molecules are administered to a subject to treat a disease associated with RAN proteins (e.g., characterized by an expansion of a nucleic acid repeat (e.g., associated with a repeat associated non-ATG (RAN) translation)). For example, in some embodiments, a subject is administered 2, 3, 4, 5, 6, 7, 8, 9, or 10 therapeutic agents (e.g., proteins, nucleic acids, small molecules, etc., or any combination thereof).

### Monoclonal Antibodies

Various aspects of the disclosure relate to antibodies and antigen-binding fragments that specifically bind to RAN proteins, and methods of making and using the same. In some embodiments, the antibody or antigen binding fragment specifically binds poly(Glycine-Alanine) [poly(GA)] proteins.

An “antibody,” as used herein, broadly refers to an immunoglobulin molecule or any functional mutant, variant, or derivation thereof. It is desired that functional mutants, variants, and derivations thereof, as well as antigen-binding fragments, retain the essential epitope binding features of an Ig molecule. Antibodies are capable of specific binding to a target through at least one antigen recognition site, located in the variable region of the immunoglobulin molecule. Generally, an intact or full-length antibody comprises two heavy chains and two light chains. Each heavy chain contains a heavy chain variable region (VH) and a first, second and third constant regions (CH1, CH2 and CH3). Each light chain contains a light chain variable region (VL) and a constant region (CL). The VH and VL regions can be further subdivided into regions of hypervariability, termed complementarity determining regions (CDR), interspersed with regions that are more conserved, termed framework regions (FR). CDR constituents on the heavy chain are referred to as CDRH1, CDRH2, and CDRH3, while CDR constituents on the light chain are referred to as CDRL1, CDRL2, and CDRL3.

The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops, or combinations of any of these methods.

Each VH and VL is composed of three CDRs and four FRs, arranged from amino-terminus to carboxy-terminus in the following order: FR1, CDR1, FR2, CDR2, FR3, CDR3, FR4. A full-length antibody can be an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The term “antigen-binding fragment” refers to any derivative of an antibody which is less than full-length, and that can bind specifically to a target. Preferably, antigen-binding fragments provided herein retain the ability to specifically bind to RAN protein, or a portion thereof. An antigen-binding fragment may comprise the heavy chain variable region (VH), the light chain variable region (VL), or both. Each of the VH and VL typically contains three complementarity determining regions CDR1, CDR2, and CDR3.

Examples of antigen binding fragments include, but are not limited to, Fab, Fab′, F(ab′)2, scFv, Fv, dsFv, diabody, affibodies, and Fd fragments. Antigen binding fragments may be produced by any appropriate means. For instance, an antigen binding fragment may be enzymatically or chemically produced by fragmentation of an intact antibody, or it may be recombinantly produced from a gene encoding the partial antibody sequence. Alternatively, an antigen binding fragment may be wholly or partially synthetically produced. An antigen binding fragment may optionally be a single chain antibody fragment. Alternatively, a fragment may comprise multiple chains which are linked together, for instance, by disulfide linkages. An antigen binding fragment may also optionally be a multimolecular complex. A functional antigen binding fragment will typically comprise at least about 50 amino acids and more typically will comprise at least about 200 amino acids.

Single-chain Fvs (scFvs) are recombinant antigen binding fragments consisting of only the variable light chain (VL) and variable heavy chain (VH) covalently connected to one another by a polypeptide linker. Either VL or VH may be the NH2-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility. ScFvs are encompassed within the term “antigen-binding fragment.”

Diabodies are dimeric scFvs. The components of diabodies typically have shorter peptide linkers than most scFvs, and they show a preference for associating as dimers (see, e.g., Holliger, P., et al. (1993) Proc. Natl. Acad. Sci. USA 90:6444-6448; Poljak, R. J., et al. (1994) Structure 2: 1121-1123). Diabodies are also encompassed within the term “antigen-binding fragment.”

A Fv fragment is an antigen binding fragment which consists of one VH and one VL domain held together by noncovalent interactions. Although the two domains of the Fv fragment, VL and VH, can be coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. The term dsFv is used herein to refer to an Fv with an engineered intermolecular disulfide bond to stabilize the VH-VL pair. dsFvs are also encompassed within the term “antigen-binding fragment.”

A F(ab′)2 fragment is an antigen binding fragment essentially equivalent to that obtained from immunoglobulins (typically IgG) by digestion with an enzyme pepsin at pH 4.0-4.5. The fragment may be recombinantly produced. F(ab′)2 are also encompassed within the term “antigen-binding fragment.”

A Fab fragment is an antigen binding fragment essentially equivalent to that obtained by reduction of the disulfide bridge or bridges joining the two heavy chain pieces in the F(ab′)2 fragment. The Fab′ fragment may be recombinantly produced. Fab′ are also encompassed within the term “antigen-binding fragment.”

A Fab fragment is an antigen binding fragment essentially equivalent to that obtained by digestion of immunoglobulins (typically IgG) with the enzyme papain. The Fab fragment may be recombinantly produced. The heavy chain segment of the Fab fragment is the Fd piece. Fab fragments are also encompassed within the term “antigen-binding fragment.”

An affibody is a small protein comprising a three-helix bundle that functions as an antigen binding molecule (e.g., an antibody mimetic). Generally, affibodies are approximately 58 amino acids in length and have a molar mass of approximately 6 kDa. Affibody molecules with unique binding properties are acquired by randomization of 13 amino acids located in two alpha-helices involved in the binding activity of the parent protein domain. Specific affibody molecules binding a desired target protein can be isolated from pools (libraries) containing billions of different variants, using methods such as phage display. Affibodies are also encompassed within the term “antigen-binding fragment.”

The term “human antibody” refers to antibodies having variable and constant regions corresponding substantially to, or derived from, antibodies obtained from human subjects, e.g., encoded by human germline immunoglobulin sequences or variants thereof. Human antibodies may include one or more amino acid residues not encoded by human germline immunoglobulin sequences (e.g., mutations introduced by random or site-specific mutagenesis in vitro or by somatic mutation in vivo). Such mutations may present in one or more of the CDRs, particularly CDR3, or in one or more of the framework regions. In some embodiments, the human antibodies may have at least one, two, three, four, five, or more positions replaced with an amino acid residue that is not encoded by the human germline immunoglobulin sequence. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29: 128-145; Hoogenboom H., and Chames P. (2000) Immunology Today 21:371-378), antibodies isolated from an animal (e.g., a mouse) that is transgenic for human immunoglobulin genes (see, e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions as defined above. In certain embodiments, however, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies may be sequences that, while derived from and related to human germline VH and VL sequences, may not naturally exist within the human antibody germline repertoire in vivo.

It should be appreciated that, in some embodiments, the disclosure contemplates variants (e.g., homologs) of amino acid and nucleic acid sequences for the heavy chain variable region and light chain variable region of the antibodies. “Homology” refers to the percent identity between two polynucleotides or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. For example, in some embodiments, nucleic acid sequences sharing substantial homology are at least 90%, at least 91%, at least 92%, at least 93%, at least 94%, at least 95%, at least 96%, at least 97%, at least 98%, or at least 99% identical in sequence. When referring to a polypeptide, or fragment thereof, the term “substantial homology” indicates that, when optimally aligned with appropriate gaps, insertions or deletions with another polypeptide, there is amino acid sequence identity in about 90 to 100% of the aligned sequences. The term “highly conserved” means at least 80% identity, preferably at least 90% identity, and more preferably, over 97% identity. For example, in some embodiments, highly conserved proteins share at least 85%, at least 90%, at least 91%, at least 92%, at least 93%, at least 94%, at least 95%, at least 96%, at least 97%, at least 98%, or at least 99% sequence identity. In some cases, highly conserved may refer to 100% identity. Identity is readily determined by one of skill in the art by, for example, the use of algorithms and computer programs known by those of skill in the art.

In some embodiments, RAN antibodies of the disclosure can bind to a RAN protein with high affinity, e.g., with a Kd less than 10⁻⁷ M, 108 M, 10⁻⁹ M, 10⁻¹⁰ M, 10⁻¹¹ M or lower. For example, anti-RAN antibodies or antigen binding fragments can bind to a RAN protein with an affinity between 5 μM and 500 nM, e.g., between 50 μM and 100 nM, e.g., between 500 μM and 50 nM. The disclosure also includes antibodies or antigen binding fragments that compete with any of the antibodies described herein for binding to RAN proteins and that have an affinity of 50 nM or lower (e.g., 20 nM or lower, 10 nM or lower, 500 μM or lower, 50 μM or lower, or 5 μM or lower). The affinity and binding kinetics of the anti-RAN protein antibody can be tested using any method known in the art including but not limited to biosensor technology (e.g., OCTET or BIACORE).

Anti-RAN antibodies may be used to treat, or assist in the treatment of, one or more symptoms of a disease associated with RAN proteins. In some embodiments, the disease associated with RAN proteins is Alzheimer's Disease (AD). In some embodiments, the RAN protein-associated disease is amyotrophic lateral sclerosis (ALS).

In some embodiments, the anti-RAN antibodies may be used to treat, or assist in the treatment of, one or more symptoms of a disease associated with RAN proteins, for example by administering a therapeutically effective amount of one or more anti-RAN antibodies to a subject diagnosed as having one or more symptoms of a disease associated with RAN proteins (e.g., the early stages of Alzheimer's disease) or being at risk of developing a disease associated with RAN proteins (e.g., based on one or more assays described in this application). In some embodiments, one or more of the anti-RAN antibody or antigen binding fragments disclosed herein are administered to a subject, wherein the subject has been characterized as having a disease associated with RAN proteins (e.g., AD) by the detection of at least one RAN protein (e.g., a poly-GA RAN protein) in a biological sample obtained from the subject.

### Anti-RAN Antibody Production

Typically, polyclonal antibodies are produced by inoculation of a suitable mammal, such as a mouse, rabbit or goat. An antigen is injected into the mammal. This induces the B-lymphocytes to produce IgG immunoglobulins specific for the antigen. This polyclonal IgG is purified from the mammal's serum. Monoclonal antibodies are generally produced by a single cell line (e.g., a hybridoma cell line). In some embodiments, an anti-RAN antibody is purified (e.g., isolated from serum).

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256: 495-499) in accordance with known methods. Hybridomas formed in this manner are then screened using standard methods, such as enzyme-linked immunosorbent assay (ELISA; e.g., RCA-based ELISA or rtPCR-based ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, any variants or fragments thereof. One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228: 1315-1317; Clackson et al. (1991) Nature, 352: 624-628; Marks et al. (1991) J. Mol. Biol., 222: 581-597; WO 92/18619; WO 91/17271; WO 92/20791; WO 92/15679; WO 93/01288; WO 92/01047; WO 92/09690; and WO 90/02809.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison et al., Proc. Natl. Acad. Sci. U.S.A. 81:6851, 1985; Takeda et al., Nature 314:452, 1985, Cabilly at al., U.S. Pat. No. 4,816,567; Boss et al., U.S. Pat. No. 4,816,397; European Patent Publication EP171496; European Patent Publication 0173494; and United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals are within the scope of the invention (see, e.g., Green et al. (1994) Nature Genetics 7, 13; and U.S. Pat. Nos. 5,545,806 and 5,569,825). For additional antibody production techniques, see, Antibodies: A Laboratory Manual, Second Edition. Edited by Edward A. Greenfield, Dana-Farber Cancer Institute, ©2014. The present disclosure is not necessarily limited to any particular source, method of production, or other special characteristics of an antibody.

Some aspects of the present disclosure relate to isolated cells (e.g., host cells) transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies. Antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Furthermore, transgenic animals, preferably mammals, comprising the aforementioned host cells may be used for the large scale production of the antibody or antibody fragments.

The transformed host cells can be grown in fermenters and cultured according to techniques known in the art to achieve optimal cell growth. Once expressed, the whole antibodies, their dimers, individual light and heavy chains, other immunoglobulin forms, or antigen binding fragments can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As used herein, “hybridoma cell” refers to an immortalized cell derived from the fusion of B lymphoblasts with a myeloma fusion partner. For preparing monoclonal antibody-producing cells (e.g., hybridoma cells), an individual animal whose antibody titer has been confirmed (e.g., a mouse) is selected, and 2 days to 5 days after the final immunization its spleen or lymph node is harvested and antibody-producing cells contained therein are fused with myeloma cells to prepare the desired monoclonal antibody-producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Kochler and Milstein (Nature 256:495 (1975)). As a fusion promoter, for example, polyethylene glycol (PEG) or Sendai virus (HVJ) is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (e.g., PEG 1000-PEG 6000) is added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier, an anti-immunoglobulin antibody (if mouse cells are used in cell fusion, anti-mouse immunoglobulin antibody is used), Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. If desired, the heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA, synthetically produced DNA or RNA, or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription, such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and are well known in the art. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains, or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus may be used for delivery of the polynucleotides or vector into a targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

### Modifications

Some aspects of the disclosure relate to antibody-drug conjugates targeted against one or more RAN proteins. As used herein, “antibody drug conjugate” refers to molecules comprising an antibody, or antigen binding fragment thereof, linked to a targeted molecule (e.g., a biologically active molecule, such as a therapeutic molecule, and/or a detectable label). Accordingly, in some embodiments, antibodies or antigen binding fragments of the disclosure may be modified with a detectable label, including, but not limited to, an enzyme, prosthetic group, fluorescent material, luminescent material, bioluminescent material, radioactive material, positron emitting metal, nonradioactive paramagnetic metal ion, and affinity label for detection and isolation of one or more RAN proteins. The detectable substance may be coupled or conjugated either directly to the polypeptides of the disclosure or indirectly, through an intermediate (such as, for example, a linker known in the art) using techniques known in the art. Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I) carbon (¹⁴C), sulfur (³⁵S), tritium (³H), indium (¹¹⁵mIn, ¹¹³mh, ¹¹²In ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, Lu, ¹⁵⁹Gd, ¹⁴⁹Pm, ¹⁴⁰La, ¹⁷⁵Yb, ¹⁶⁶Ho, ⁹⁰Y⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, and tin (¹¹³Sn, ¹¹⁷Sn). The detectable substance may be coupled or conjugated either directly to the anti-RAN antibodies or antigen-binding fragments of the disclosure or indirectly, through an intermediate (such as, for example, a linker known in the art) using techniques known in the art. Anti-RAN antibodies conjugated to a detectable substance may be used for diagnostic assays as described herein.

In some embodiments, antibodies or antigen binding fragments of the disclosure may be modified with a therapeutic moiety (e.g., therapeutic agent). In some embodiments, the antibody is coupled to the targeted agent via a linker. As used herein, the term “linker” refers to a molecule or sequence, such as an amino acid sequence, that attaches, as in a bridge, one molecule or sequence to another molecule or sequence. “Linked,” “conjugated,” or “coupled” means attached or bound by covalent bonds, or non-covalent bonds, or other bonds, such as van der Waals forces. Antibodies described by the disclosure can be linked to the targeted agent (e.g., therapeutic moiety or detectable moiety) directly, e.g., as a fusion protein with protein or peptide detectable moieties (with or without an optional linking sequence, e.g., a flexible linker sequence) or via a chemical coupling moiety. A number of such coupling moieties are known in the art, e.g., a peptide linker or a chemical linker, e.g., as described in International Patent Application Publication No. WO 2009/036092. In some embodiments, the linker is a flexible amino acid sequence. Examples of flexible amino acid sequences include glycine and serine rich linkers, which comprise a stretch of two or more glycine residues. In some embodiments, the linker is a photolinker. Examples of photolinkers include ketyl-reactive benzophenone (BP), anthraquinone (AQ), nitrene-reactive nitrophenyl azide (NPA), and carbene-reactive phenyl-(trifluoromethyl)diazirine (PTD).

### Pharmaceutical Compositions

In some aspects, the disclosure relates to pharmaceutical compositions comprising anti-RAN antibodies or antigen binding fragments. In some embodiments, the composition comprises an anti-RAN antibody and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described below. The active ingredients may be admixed or compounded with any conventional, pharmaceutically acceptable carrier or excipient. The compositions may be sterile.

Typically, pharmaceutical compositions are formulated for delivering an effective amount of an agent (e.g., an anti-RAN antibody). In general, an “effective amount” of an active agent refers to an amount sufficient to elicit the desired biological response (e.g., ameliorating one or more symptoms of Alzheimer's disease). An effective amount of an agent may vary depending on such factors as the desired biological endpoint, the pharmacokinetics of the compound, the disease being treated (e.g., Alzheimer's disease, repeat expansion diseases), the mode of administration, and the patient.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18th Ed. (1990).

It will be understood by those skilled in the art that any mode of administration, vehicle or carrier conventionally employed and which is inert with respect to the active agent may be utilized for preparing and administering the pharmaceutical compositions of the present disclosure. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the disclosure, will experience no difficulty in determining suitable and appropriate vehicles, excipients and carriers or in compounding the active ingredients therewith to form the pharmaceutical compositions of the disclosure.

An effective amount, also referred to as a therapeutically effective amount, of a compound (e.g., an anti-RAN antibody) is an amount sufficient to ameliorate at least one adverse effect associated with a disease associated with RAN proteins, such as, e.g., memory loss, cognitive impairment, loss of coordination, speech impairment, etc. In some embodiments, the neurological disease associated with RAN proteins is Alzheimer's Disease (AD). In some embodiments, the RAN protein-associated disease is amyotrophic lateral sclerosis (ALS). The therapeutically effective amount to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount.

Combined with the teachings provided herein, by choosing among the various active compounds and weighing factors such as potency, relative bioavailability, patient body weight, severity of adverse side-effects and selected mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered, the size of the subject, or the severity of the disease or condition. One of ordinary skill in the art can empirically determine the effective amount of a particular nucleic acid and/or other therapeutic agent without necessitating undue experimentation.

In some cases, compounds of the disclosure are prepared in a colloidal dispersion system. Colloidal dispersion systems include lipid-based systems including oil-in-water emulsions, micelles, mixed micelles, and liposomes. In some embodiments, a colloidal system of the disclosure is a liposome. Liposomes are artificial membrane vessels which are useful as a delivery vector in vivo or in vitro. It has been shown that large unilamellar vesicles (LUVs), which range in size from 0.2-4.0 m can encapsulate large macromolecules.

Liposomes may be targeted to a particular tissue by coupling the liposome to a specific ligand such as a monoclonal antibody, sugar, glycolipid, or protein. Ligands which may be useful for targeting a liposome to, for example, an smooth muscle cell include, but are not limited to: intact or fragments of molecules which interact with smooth muscle cell specific receptors and molecules, such as antibodies, which interact with the cell surface markers of cancer cells. Such ligands may easily be identified by binding assays well known to those of skill in the art. In still other embodiments, the liposome may be targeted to a tissue by coupling it to an antibody known in the art.

Compounds described by the disclosure may be administered alone (e.g., in saline or buffer) or using any delivery vehicle known in the art. For instance the following delivery vehicles have been described: cochleates; Emulsomes; ISCOMs; liposomes; live bacterial vectors (e.g., Salmonella, Escherichia coli, Bacillus Calmette-Guerin, Shigella, Lactobacillus); live viral vectors (e.g., Vaccinia, adenovirus, Herpes simplex); microspheres; nucleic acid vaccines; polymers (e.g., carboxymethylcellulose, chitosan); polymer rings; proteosomes; sodium fluoride; transgenic plants; virosomes; and, virus-like particles.

The formulations of the disclosure are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In addition to the formulations described herein, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see, Langer R (1990) Science 249:1527-1533, which is incorporated herein by reference.

The compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

### Administration

A therapeutic agent may be delivered by any suitable modality known in the art. In some embodiments, a therapeutic agent (e.g., a protein, antibody, interfering nucleic acid, etc.) is delivered to a subject by a vector, such as a viral vector (e.g., adenovirus vector, recombinant adeno-associated virus vector (rAAV vector), lentiviral vector, etc.) or a plasmid-based vector. In some embodiments, a therapeutic agent is delivered to a subject (e.g., a subject having Alzheimer's disease characterized by expression of one or more RAN proteins) in a recombinant adeno-associated virus (rAAV) particle.

In some embodiments, a recombinant rAAV particle comprises a nucleic acid vector, such as a single-stranded (ss) or self-complementary (sc) AAV nucleic acid vector. In some embodiments, the nucleic acid vector comprises a transgene encoding an therapeutic agent as described herein (e.g., a protein, antibody, interfering nucleic acid, etc.), and one or more regions comprising inverted terminal repeat (ITR) sequences (e.g., wild-type ITR sequences or engineered ITR sequences) flanking the expression construct. In some embodiments, the nucleic acid is encapsidated by a viral capsid. In some embodiments, the transgene is operably linked to a promoter, for example a constitutive promoter or an inducible promoter. In some embodiments, the promoter is a tissue-specific (e.g., CNS-specific) promoter. In some embodiments, a rAAV particle comprises a viral capsid that has a tropism for CNS tissue, for example AAV9 capsid protein or AAV.PHPB capsid protein.

Aspects of the disclosure relate to the delivery of a therapeutically effective amount of a therapeutic agent to a subject. In some embodiments, a therapeutically effective amount is an amount effective in reducing repeat expansions in the subject. In some embodiments, a therapeutically effective amount is an amount effective in reducing the transcription of RNAs that produce RAN proteins in a subject. In certain embodiments, a therapeutically effective amount is an amount effective in reducing the translation of RAN proteins in a subject. In some embodiments, a therapeutically effective amount is an amount effective for treating Alzheimer's disease associated with repeat expansions. “Reducing” expression of a repeat sequence or RAN protein translation refers to a decrease in the amount or level of repeat sequence expression or RAN protein translation in a subject after administration of a therapeutic agent (and relative to the amount or level in the subject prior to the administration).

In certain embodiments, the effective amount is an amount effective in reducing the level of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative to the level of RAN proteins in a cell or subject that has not been administered a therapeutic agent). In certain embodiments, the effective amount is an amount effective in reducing the translation of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent).

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor*, alcohols, oils, modified oils, glycols, polysorbates, cyclodextrins, polymers, and mixtures thereof. The exemplary liquid dosage forms in certain embodiments are formulated for ease of swallowing, or for administration via feeding tube.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Therapeutic agents described herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

A therapeutic agent can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a therapeutic agent required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, eight months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Routes of administration include but are not limited to oral, parenteral, intravenous, intramuscular, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

In some embodiments, a treatment for a disease associated with RAN protein expression is administered to the central nervous system (CNS) of a subject in need thereof. As used herein, the “central nervous system (CNS)” refers to all cells and tissues of the brain and spinal cord of a subject, including but not limited to neuronal cells, glial cells, astrocytes, cerebrospinal fluid, etc. Modalities of administering a therapeutic agent to the CNS of a subject include direct injection into the brain (e.g., intracerebral injection, intraventricular injection, intraparenchymal injection, etc.), direct injection into the spinal cord of a subject (e.g., intrathecal injection, lumbar injection, etc.), or any combination thereof.

In some embodiments, a treatment as described by the disclosure is systemically administered to a subject, for example by intravenous injection. Systemically administered therapeutic molecules can be modified, in some embodiments, in order to improve delivery of the molecules to the CNS of a subject. Examples of modifications that improve CNS delivery of therapeutic molecules include but are not limited to co-administration or conjugation to blood brain barrier-targeting agents (e.g., transferrin, melanotransferrin, low-density lipoprotein (LDL), angiopeps, RVG peptide, etc., as disclosed by Georgieva et al. Pharmaceuticals 6(4): 557-583 (2014)), coadministration with BBB disrupting agents (e.g., bradykinins), and physical disruption of the BBB prior to administration (e.g., by MRI-Guided Focused Ultrasound), etc.

The following Example is intended to illustrate the benefits of the present invention and to describe particular embodiments, but are not intended to exemplify the full scope of the invention. Accordingly, it will be understood that the Example is not meant to limit the scope of the invention.

### Example

This example describes investigating translation of poly-Glycine-Alanine (poly-GA) repeat-associated non-ATG proteins in the context of Alzheimer's disease (AD) patients.

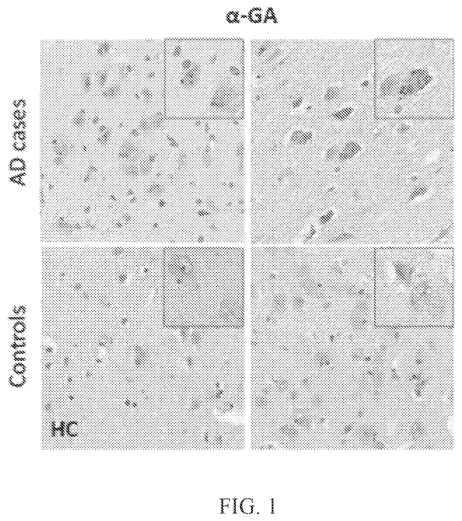

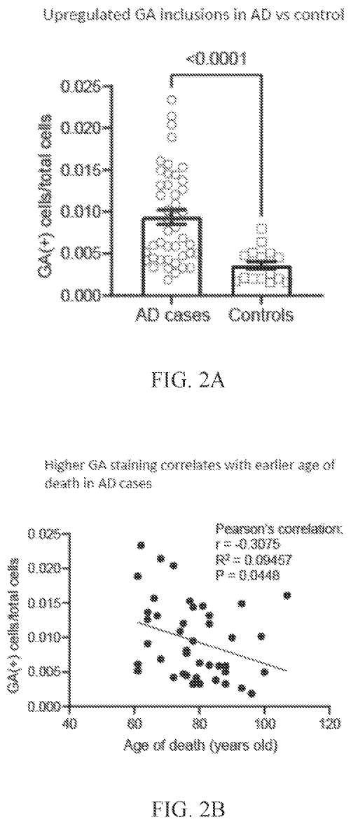

Immunohistochemistry and staining of AD patient tissues was performed using a poly-GA-repeat protein specific antibody. FIG. 1 shows representative data indicating that poly-GA RAN proteins accumulate in Alzheimer's disease (AD) tissue. The effects of poly-GA translation, accumulation, and aggregation were also investigated. FIG. 2A shows data indicating that poly-GA inclusions are up-regulated in AD tissue relative to control (e.g., non-AD) tissue. FIG. 2B shows data indicating that higher poly-GA staining correlates with an earlier age of death in AD patients.

The repeat motifs of the putative AD RAN proteins were used to identify all possible DNA sequences that could encode poly-GA RAN proteins. For example, all possible repeat motifs encoding GA are shown below in Table 1.

TABLE 1
All possible nucleic acid sequences encoding GA

| | | | | |
|---|---|---|---|---|
| GA coding motifs | GGTGCT | GGTGCC | GGTGCA | GGTGCG |
| | GGCGCT | GGCGCC | GGCGCA | GGCGCG |
| | GGAGCT | GGAGCC | GGAGCA | GGAGCG |
| | GGGGCT | GGGGCC | GGGGCA | GGGGCG |

These sequences were used to design fluorophore-conjugated DNA probes that target all possible repeat motifs that could encode a given candidate RAN AD protein. Fluorescence in situ hybridization (FISH) screening of frozen AD brains using the RAN protein nucleic acid probes showed punctate RNA foci, a hallmark of repeat expansion diseases (FIG. 1). No similar RNA foci were found in controls or RAN-negative AD cases. Detection of RNA foci and RAN protein staining in candidate AD brain tissues demonstrates the presence of poly-GA repeat expansion mutations in AD.

To identify a specific locus containing the repeat expansion mutations in RAN and RNA foci positive candidate AD cases, a pull-down assay was used to enrich the specific repeat expansion mutation and the corresponding flanking sequences using a biotin-tagged nuclease-deficient Cas9 (dCas9) approach. This dCas9-based enrichment tool pulls down and enriches specific DNA sequences by taking advantage of the rapid kinetics and high stability of single guide RNA/dCas9 (sgRNA-dCas9) complexes without the need to denature target DNA. Expanded repeats provide multiple binding sites for sgRNAs, thus increasing the probability of interaction between sgRNA-dCas9 complexes and expanded repeats compared to shorter repeat tracts.

In some embodiments, a poly-GA repeat RAN protein is translated from an mRNA transcript encoded by a genetic locus or one or more genes (e.g., is encoded by one or more genes) set forth in Table 2. In some embodiments, a poly-GA repeat RAN protein is translated from an mRNA transcript encoded by a chromosomal locus in chrX beginning at 100748986 and ending at 100749205. In some embodiments, a poly-GA repeat RAN protein is translated from an mRNA transcript encoded by ARMCX4.

TABLE 2
Poly-GA-encoding chromosomal loci and gene candidates

| Chr | Start | End | Gene Name |
|---|---|---|---|
| chr1 | 10689951 | 10690382 | PEX14 |
| chr1 | 43996518 | 43996909 | PTPRF |
| chr1 | 229568225 | 229568424 | ACTA1 |
| chr1 | 225052763 | 225053047 | DNAH14 |
| chr1 | 144593548 | 144593837 | PFN1P2 |
| chr1 | 156405445 | 156405644 | C1orf61 |
| chr1 | 27854229 | 27854570 | WASF2 |
| chr1 | 249240409 | 249240608 | PGBD2 |
| chr1 | 148555840 | 148556039 | NBPF15 |
| chr1 | 9890 | 10089 | DDX11L1 |
| chr1 | 91013321 | 91013724 | BARHL2 |
| chr1 | 26882257 | 26882456 | MIR1976 |
| chr1 | 10716625 | 10716970 | CASZ1 |
| chr1 | 95320359 | 95320558 | SLC44A3 |
| chr1 | 236332250 | 236332843 | GPR137B |
| chr1 | 204043901 | 204044188 | SOX13 |
| chr1 | 17265730 | 17265929 | CROCC |
| chr1 | 201963098 | 201963297 | RNPEP |
| chr1 | 180332654 | 180333021 | MIR3121 |
| chr1 | 161279805 | 161280004 | MPZ |
| chr1 | 95285699 | 95285898 | SLC44A3 |
| chr1 | 150552141 | 150552575 | MCL1 |
| chr1_gl000192_random | 482701 | 482900 | HYDIN2 |
| chr1_gl000192_random | 482346 | 482545 | HYDIN2 |
| chr10 | 71892369 | 71892728 | AIFM2 |
| chr10 | 130989477 | 130989878 | MGMT |
| chr10 | 133579203 | 133579402 | LINC01164 |
| chr10 | 134938083 | 134938476 | KNDC1 |
| chr10 | 62408347 | 62408883 | ANK3 |
| chr10 | 21824084 | 21824283 | MLLT10 |
| chr10 | 96163284 | 96163483 | TBC1D12 |
| chr10 | 77732564 | 77732763 | LRMDA |
| chr10 | 35645973 | 35646186 | CCNY |
| chr10 | 45675760 | 45675985 | MIR3156-1 |
| chr10 | 135480145 | 135480344 | DUX4L2 |
| chr10 | 49239612 | 49239950 | AGAP12P |
| chr10 | 50874040 | 50874239 | C10orf53 |
| chr11 | 6411774 | 6412135 | SMPD1 |
| chr11 | 1774885 | 1775331 | IFITM10 |
| chr11 | 116396856 | 116397100 | BUD13 |
| chr11 | 44748624 | 44748823 | TSPAN18 |
| chr11 | 44615612 | 44616069 | CD82 |
| chr11 | 63788800 | 63788999 | OTUB1 |
| chr11 | 71184261 | 71184671 | NADSYN1 |
| chr11 | 118773724 | 118774112 | MIR4492 |
| chr11 | 910772 | 910971 | CHID1 |
| chr12 | 54552767 | 54553105 | SMUG1 |
| chr12 | 46004941 | 46005424 | LINC00938 |
| chr12 | 95529 | 95728 | |
| chr12 | 131834684 | 131834883 | LINC01257 |
| chr12 | 129078513 | 129078712 | SLC15A4 |
| chr12 | 50467966 | 50468434 | ASIC1 |
| chr12 | 2098368 | 2098761 | DCP1B |
| chr12 | 83080647 | 83081023 | TMTC2 |
| chr12 | 53440806 | 53441005 | TNS2 |
| chr12 | 54476279 | 54476698 | LOC100240735 |
| chr13 | 112722260 | 112722635 | SOX1 |
| chr13 | 21562515 | 21562819 | LATS2 |
| chr13 | 111213761 | 111213960 | RAB20 |
| chr13 | 19345023 | 19345462 | ANKRD20A9P |
| chr13 | 29105279 | 29105478 | FLT1 |
| chr13 | 19364346 | 19364732 | ANKRD20A9P |
| chr13 | 112851851 | 112852263 | SOX1 |
| chr13 | 50159719 | 50159918 | RCBTB1 |
| chr14 | 106146451 | 106146792 | ELK2AP |
| chr14 | 81902200 | 81902399 | STON2 |
| chr14 | 89643810 | 89644243 | FOXN3 |
| chr14 | 76279152 | 76279457 | TTLL5 |
| chr14 | 99654843 | 99655042 | BCL11B |
| chr14 | 36294641 | 36294840 | BRMS1L |
| chr15 | 67236065 | 67236264 | SMAD3 |
| chr15 | 69762527 | 69762883 | RPLP1 |
| chr15 | 40728354 | 40728553 | BAHD1 |
| chr15 | 52752769 | 52753205 | MYO5A |
| chr15 | 100545833 | 100546208 | DNM1P46 |
| chr15 | 76029971 | 76030441 | DNM1P35 |
| chr15 | 43663174 | 43663373 | TUBGCP4 |
| chr16 | 86981615 | 86981983 | C16orf95 |
| chr16 | 83984448 | 83985043 | OSGIN1 |
| chr16 | 85269536 | 85269820 | LINC00311 |
| chr16 | 12678868 | 12679067 | MIR4718 |
| chr16 | 5644938 | 5645137 | RBFOX1 |
| chr16 | 28318085 | 28318486 | SBK1 |
| chr16 | 88810953 | 88811152 | MIR4722 |
| chr16 | 88088115 | 88088385 | BANP |
| chr16 | 49526231 | 49526594 | C16orf78 |
| chr16 | 7395120 | 7395319 | RBFOX1 |
| chr16 | 88563499 | 88563828 | MIR5189 |
| chr16 | 57597346 | 57597545 | ADGRG5 |
| chr16 | 188566 | 189019 | NPRL3 |
| chr16 | 67450185 | 67450384 | ZDHHC1 |
| chr16 | 818643 | 819390 | MIR662 |
| chr17 | 79286772 | 79287032 | LINC00482 |
| chr17 | 79667442 | 79667785 | MRPL12 |
| chr17 | 34758456 | 34758784 | TBC1D3H |
| chr17 | 6128763 | 6128962 | WSCD1 |
| chr17 | 34505358 | 34505695 | TBC1D3B |
| chr17 | 36282333 | 36282548 | TBC1D3 |
| chr17 | 34809513 | 34809752 | |
| chr17 | 34593380 | 34593770 | TBC1D3C |
| chr17 | 81038575 | 81038859 | METRNL |
| chr17 | 11514114 | 11514563 | DNAH9 |
| chr17 | 7080168 | 7080367 | ASGR1 |
| chr17 | 80514358 | 80514781 | FOXK2 |
| chr17 | 45608994 | 45609450 | NPEPPS |
| chr17 | 26712254 | 26712453 | SARM1 |
| chr17 | 2594881 | 2595080 | CLUH |
| chr17 | 27407060 | 27407259 | TIAF1 |
| chr17 | 36412403 | 36412844 | LOC440434 |
| chr17 | 47307999 | 47308198 | PHOSPHO1 |
| chr17 | 80709161 | 80709360 | TBCD |
| chr18 | 14273580 | 14273999 | CYP4F35P |
| chr18 | 14450350 | 14450549 | CXADRP3 |
| chr18 | 14293215 | 14293523 | CYP4F35P |
| chr18 | 14328729 | 14329144 | CYP4F35P |
| chr18 | 14309542 | 14309888 | CYP4F35P |
| chr18 | 6956385 | 6956649 | LINC00668 |
| chr18 | 48722763 | 48722962 | MEX3C |
| chr19 | 36643288 | 36643679 | COX7A1 |
| chr19 | 50155158 | 50155544 | SCAF1 |
| chr19 | 56292478 | 56292715 | RFPL4AL1 |
| chr19 | 46264902 | 46265216 | SIX5 |
| chr19 | 2726435 | 2726634 | DIRAS1 |
| chr19 | 613577 | 613776 | POLRMT |
| chr19 | 2808349 | 2808548 | ZNF554 |
| chr19 | 878204 | 878514 | MED16 |
| chr19 | 878743 | 879118 | MED16 |
| chr19 | 38419661 | 38420011 | SIPA1L3 |
| chr19 | 2181592 | 2181896 | DOT1L |
| chr19 | 55854393 | 55854716 | KMT5C |
| chr19 | 18362416 | 18362615 | PDE4C |
| chr19 | 52800262 | 52800461 | ZNF480 |
| chr19 | 33792781 | 33792980 | CEBPA |
| chr2 | 131129836 | 131130117 | PTPN18 |
| chr2 | 43037117 | 43037406 | HAAO |
| chr2 | 91635601 | 91635800 | LOC654342 |
| chr2 | 88124968 | 88125167 | RGPD2 |
| chr2 | 135875208 | 135875407 | RAB3GAP1 |
| chr2 | 218723441 | 218723658 | TNS1 |
| chr2 | 95432093 | 95432300 | FAM95A |
| chr2 | 95435059 | 95435456 | FAM95A |
| chr20 | 61326206 | 61326612 | NTSR1 |
| chr20 | 29517854 | 29518289 | FRG1BP |
| chr20 | 25843143 | 25843342 | FAM182B |
| chr20 | 59792831 | 59793030 | CDH4 |
| chr20 | 4470132 | 4470488 | PRNP |
| chr20 | 25847345 | 25847544 | FAM182B |
| chr20 | 60186097 | 60186371 | CDH4 |
| chr20 | 60476254 | 60476624 | MIR1257 |
| chr20 | 60909810 | 60910009 | MIR4758 |
| chr20 | 61436635 | 61436978 | OGFR |
| chr20 | 35982776 | 35982975 | SRC |
| chr20 | 61448252 | 61448451 | COL9A3 |
| chr20 | 62601327 | 62601526 | ZNF512B |
| chr21 | 46438691 | 46438890 | PICSAR |
| chr21 | 44854550 | 44854749 | SIK1 |
| chr21 | 15248181 | 15248585 | CYP4F29P |
| chr21 | 15225886 | 15226200 | CYP4F29P |
| chr21 | 46421692 | 46421996 | PICSAR |
| chr21 | 42792115 | 42792577 | MX1 |
| chr21 | 9826211 | 9826410 | |
| chr22 | 34729102 | 34729477 | LARGE1 |
| chr22 | 50142398 | 50142739 | |
| chr22 | 50322997 | 50323353 | CRELD2 |
| chr22 | 45665111 | 45665439 | UPK3A |
| chr22 | 42926279 | 42926478 | RRP7A |
| chr22 | 46148395 | 46148669 | MIR4762 |
| chr22 | 51113309 | 51113508 | SHANK3 |
| chr22 | 42310916 | 42311475 | SHISA8 |
| chr22 | 20137949 | 20138148 | CCDC188 |
| chr22 | 39239966 | 39240165 | NPTXR |
| chr3 | 40832765 | 40832997 | ZNF621 |
| chr3 | 127110873 | 127111235 | TPRA1 |
| chr3 | 196699904 | 196700281 | PIGZ |
| chr3 | 9610871 | 9611337 | LHFPL4 |
| chr3 | 127257319 | 127257773 | TPRA1 |
| chr3 | 190825323 | 190825681 | OSTN |
| chr3 | 115472357 | 115472974 | GAP43 |
| chr3 | 50526997 | 50527271 | CACNA2D2 |
| chr3 | 195600021 | 195600385 | TNK2 |
| chr3 | 13061650 | 13061849 | IQSEC1 |
| chr3 | 9066494 | 9066926 | RAD18 |
| chr3 | 122399994 | 122400431 | PARP14 |
| chr3 | 126721895 | 126722296 | PLXNA1 |
| chr3 | 50712828 | 50713240 | DOCK3 |
| chr4 | 191044059 | 191044258 | DUX4L8 |
| chr4 | 133025391 | 133025899 | PCDH10 |
| chr4 | 122159052 | 122159251 | TNIP3 |
| chr4 | 2388768 | 2388972 | ZFYVE28 |
| chr4 | 166247942 | 166248141 | MSMO1 |
| chr4 | 125633799 | 125633998 | ANKRD50 |
| chr5 | 176536945 | 176537174 | FGFR4 |
| chr5 | 3590364 | 3590739 | IRX1 |
| chr5 | 16428038 | 16428340 | ZNF622 |
| chr5 | 136841617 | 136841816 | SPOCK1 |
| chr5 | 11571 | 11770 | PLEKHG4B |
| chr5 | 169715127 | 169715326 | LCP2 |
| chr5 | 176798008 | 176798318 | SLC34A1 |
| chr5 | 139061582 | 139061781 | CXXC5 |
| chr5 | 149217457 | 149217656 | PPARGC1B |
| chr5 | 175621835 | 175622061 | LOC643201 |
| chr5 | 131563677 | 131563897 | P4HA2 |
| chr6 | 169251764 | 169251963 | THBS2 |
| chr6 | 108254708 | 108254907 | SEC63 |
| chr6 | 74363038 | 74363326 | SLC17A5 |
| chr6 | 42973984 | 42974183 | MEA1 |
| chr6 | 72596035 | 72596333 | RIMS1 |
| chr6 | 157098164 | 157098363 | ARID1B |
| chr6 | 157099861 | 157100060 | ARID1B |
| chr7 | 151577853 | 151578052 | PRKAG2 |
| chr7 | 155260363 | 155260618 | EN2 |
| chr7 | 8466976 | 8467175 | NXPH1 |
| chr7 | 151038972 | 151039171 | NUB1 |
| chr7 | 154174203 | 154174402 | DPP6 |
| chr7 | 101270813 | 101271012 | MYL10 |
| chr7 | 66021139 | 66021338 | GS1-124K5.11 |
| chr7 | 87104869 | 87105068 | ABCB4 |
| chr8 | 145738504 | 145738703 | MFSD3 |
| chr8 | 55372114 | 55372447 | SOX17 |
| chr8 | 98616282 | 98616764 | MTDH |
| chr8 | 67291496 | 67291704 | RRS1-AS1 |
| chr8 | 59466078 | 59466322 | SDCBP |
| chr8 | 25057258 | 25057510 | DOCK5 |
| chr8 | 145155424 | 145155722 | SHARPIN |
| chr8 | 143287396 | 143287631 | LINC00051 |
| chr8 | 133686271 | 133686492 | LRRC6 |
| chr8 | 144660449 | 144660648 | NAPRT |
| chr9 | 100615977 | 100616176 | FOXE1 |
| chr9 | 139912174 | 139912512 | C9orf139 |
| chr9 | 45732495 | 45732876 | |
| chr9 | 44876231 | 44876601 | FAM27C |
| chr9 | 67339502 | 67339701 | AQP7P1 |
| chr9 | 44882651 | 44882888 | FAM27C |
| chr9 | 82974369 | 82974703 | TLE4 |
| chr9 | 133025859 | 133026058 | NCS1 |
| chr9 | 67786941 | 67787211 | FAM27B |
| chr9 | 82478403 | 82478732 | TLE4 |
| chr9 | 44997256 | 44997471 | FAM27C |
| chr9 | 132331439 | 132331922 | C9orf50 |
| chr9 | 132585479 | 132585807 | TOR1A |
| chr9 | 140411598 | 140411797 | PNPLA7 |
| chr9 | 20365529 | 20365959 | MIR4473 |
| chr9 | 132428409 | 132428678 | PRRX2 |
| chr9 | 124493886 | 124494148 | DAB2IP |
| chr9 | 27573441 | 27573859 | C9orf72 |
| chr9 | 139222229 | 139222428 | GPSM1 |
| chrUn_gl000212 | 77505 | 77953 | FAM230C |
| chrUn_gl000220 | 122042 | 122241 | RNA5-8SN5 |
| chrX | 24329554 | 24330097 | SUPT20HL2 |
| chrX | 24382256 | 24382455 | SUPT20HL1 |
| chrX | 100748986 | 100749205 | ARMCX4 |
| chrX | 71996646 | 71997015 | FAM236A |
| chrX | 153618674 | 153618873 | RPL10 |
| chrX | 153170676 | 153171221 | AVPR2 |
| chrX | 9754235 | 9754434 | SHROOM2 |
| chrX | 72163493 | 72163825 | FAM226A |

Additional data was collected which further characterized the polyGA pathology described herein, and identified a genetic mutation that could cause polyGA pathology in AD.

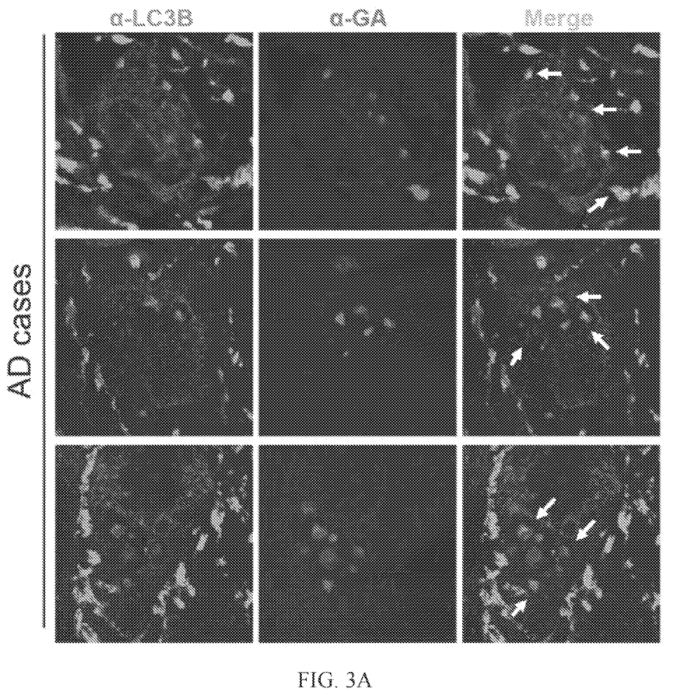

Using double immunofluorescence (IF), it is demonstrated herein that polyGA aggregates colocalize with LC3B3 and LAMP1 in the hippocampal regions of AD autopsy brains, but not in controls (FIGS. 3A and 3B). LC3B3 and LAMP1 are the makers of autophagosomes and lysosomes, respectively. This result demonstrates that polyGA aggregates are associated with early autophagic events, and suggests that polyGA RAN proteins are toxic to cells.

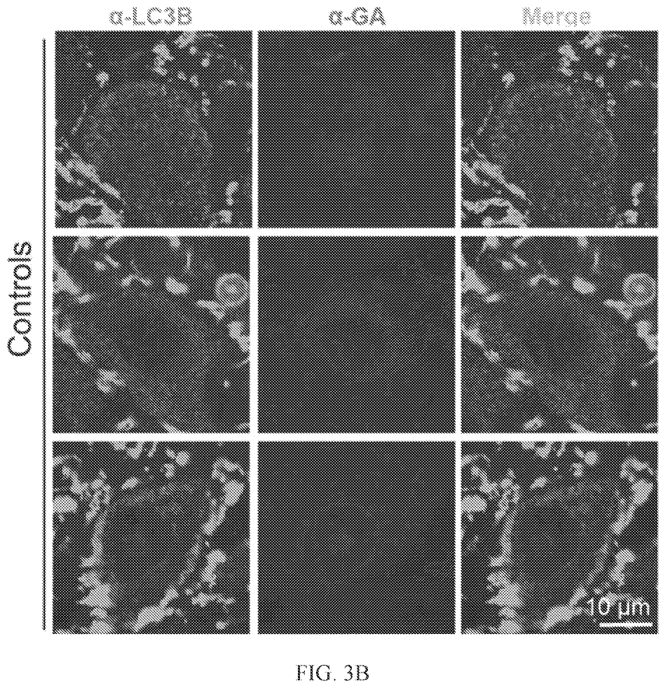

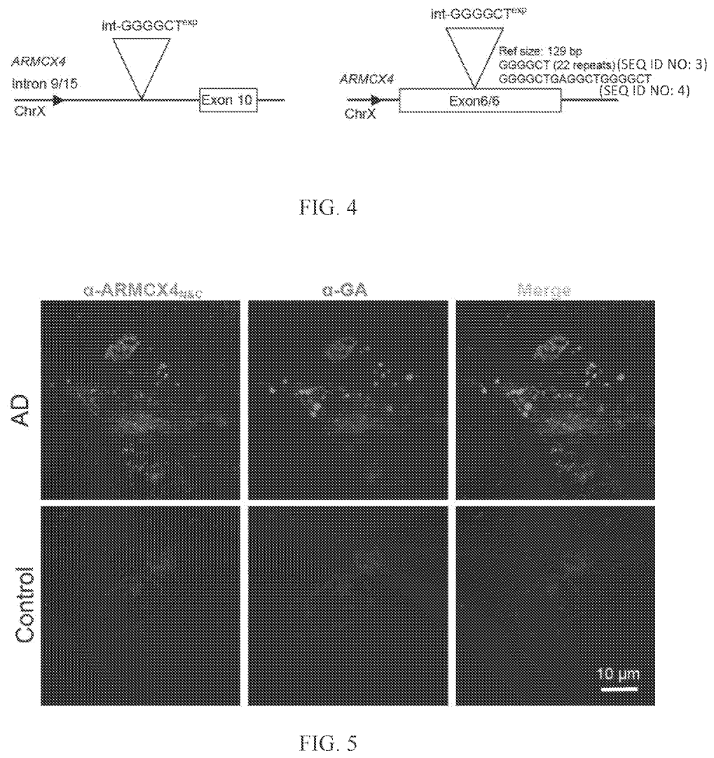

To identify the mutations that could cause polyGA aggregates in AD and ALS, CRISPR deactivated Cas9 repeat enrichment and detection experiments were performed using single guide RNAs that target GA-encoding repeats, as described above. A repeat expansion in the ARMCX4 gene was identified that encodes GA-rich proteins (FIG. 4). Using commercialized antibodies targeting ARMCX4 proteins, co-localization of ARMCX4 proteins with polyGA aggregates was detected in the hippocampal regions in AD autopsy brains, but not controls (FIG. 5).

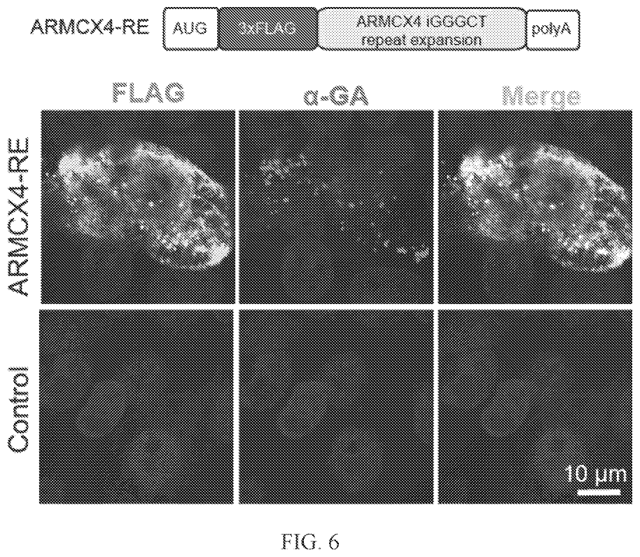

To test if an anti-GA antibody recognizes GA-rich proteins expressed from the ARMCX4 repeat expansion, ARMCX4-RE plasmids were designed and cloned in which FLAG tag expresses in frame with ARMCX4 GA-rich proteins. In ARMCX4-RE overexpressing HEK293T cells, anti-GA antibody staining co-localizes with FLAG tag. No similar staining was detected in cells transfected with control plasmids. The staining pattern of anti-GA antibody in HEK293T cells transfected with ARMCX4-RE plasmids is similar to the polyGA aggregate staining detected in AD autopsy brains (FIG. 6).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method for treating a subject having or suspected of having Alzheimer's disease (AD), the method comprising administering to the subject one or more therapeutic agents that reduce poly-(Glycine-Alanine) (poly-GA) repeat-associated non-ATG (RAN) protein translation, expression, aggregation, or accumulation.

2. The method of claim 1, wherein the poly-GA RAN protein is translated from an mRNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IF1TMO, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNM1P46, DNM1P35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIRI257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A.

3. The method of claim 1 or claim 2, wherein the one or more therapeutic agents comprise an anti-RAN protein antibody, an inhibitory nucleic acid, a peptide, and/or a small molecule.

4. The method of claim 3, wherein the anti-RAN protein antibody is an anti-poly-GA antibody.

5. The method of claim 4, wherein the anti-poly-GA antibody specifically binds to a poly-GA repeat region of the RAN protein in the subject.

6. The method of claim 4 or claim 5, wherein the anti-poly-GA antibody is a monoclonal antibody.

7. The method of any one of claims 1 to 3, wherein the inhibitory nucleic acid is double-stranded RNA (dsRNA), short-interfering RNA (siRNA), short-hairpin RNA (shRNA), microRNA (miRNA), artificial microRNA (amiRNA), an aptamer, or an antisense oligonucleotide (ASO).

8. The method of claim 7, wherein the inhibitory nucleic acid comprises a region of complementarity with a nucleic acid sequence encoding a poly-GA repeat expansion in the subject.

9. The method of claim 7 or claim 8, wherein the inhibitory nucleic acid comprises a region of complementarity with an RNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNMIP46, DNMIP35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A.

10. The method of any one of claims 7 to 9, wherein the inhibitory nucleic acid comprises a region of complementarity with a nucleic acid sequence present at a chromosomal locus set forth in Table 2.

11. The method of any one of claims 1 to 10, wherein the subject is a mammal.

12. The method of any one of claims 1 to 11, wherein the subject is a human.

13. The method of any one of claims 1 to 12, wherein the administration of the one or more therapeutic agents to the subject results in a reduction of poly-GA RAN protein translation, expression, aggregation or accumulation in the subject, relative to the level of poly-GA RAN protein translation, expression, aggregation or accumulation in the subject prior to the administration.

14. A method of identifying a subject as having or likely to develop Alzheimer's disease (AD), the method comprising:
(i) obtaining a biological sample from the subject;
(ii) contacting the biological sample with detectable agent specific for a poly-(Glycine-Alanine) (poly-GA) repeat-associated non-ATG (RAN) protein;
(iii) performing an assay on the biological sample to detect the presence of poly-GA RAN proteins, wherein poly-GA RAN proteins are detected via the detectable agent; and
(iv) identifying the subject as having or likely to develop AD based upon the detection of the detectable agent.

15. The method of claim 14, wherein the poly-GA RAN protein is encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNMIP46, DNMIP35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A.

16. The method of claim 14 or claim 15, wherein the subject is identified as having AD based upon the detection of the detectable agent.

17. The method of claim 14 or claim 15, wherein the subject is identified as likely to develop AD based upon the detection of the detectable agent.

18. The method of any one of claims 14 to 17, wherein the biological sample is tissue, blood, serum, or cerebrospinal fluid (CSF), optionally wherein the tissue is brain tissue or spinal cord tissue.

19. The method of any one of claims 14 to 18, wherein the assay comprises an antibody-based capture assay, binding assay, hybridization assay, immunoblot analysis, Western blot analysis, immunohistochemistry, dCas9-based enrichment, label free immunoassays, immunoquantitative PCR, mass spectrometry, bead-based immunoassays, immunoprecipitation, immunostaining, immunoelectrophoresis, and/or ELISA.

20. The method of claim 19, wherein the dCas9 protein is Streptococcus pyogenes dCas9 (spdCas9).

21. The method of claim 19, wherein the ELISA is RCA-based ELISA or rtPCR-based ELISA.

22. The method of any one of claims 14 to 21, wherein the detectable agent comprises an anti-poly-GA antibody.

23. The method of claim 22, wherein the anti-poly-GA antibody is linked to a detectable label.

24. The method of claim 23, wherein the detectable label comprises an enzyme, prosthetic group, fluorescent material, luminescent material, bioluminescent material, radioactive material, positron emitting metal, nonradioactive paramagnetic metal ion, or affinity label.

25. The method of any one of claims 14 to 24, wherein the subject is mammal.

26. The method of claim 25, wherein the subject is a human.

27. The method of any one of claims 14 to 26, wherein the subject identified as having or likely to develop AD is administered one or more therapeutic agents.

28. The method of claim 27, wherein the one or more therapeutic agents comprises an anti-RAN protein antibody, an inhibitory nucleic acid, a peptide, and/or a small molecule.

29. The method of claim 28, wherein the anti-RAN protein antibody is an anti-poly-GA antibody.

30. The method of claim 29, wherein the anti-poly-GA antibody specifically binds to a poly-GA repeat region of the RAN protein in the subject.

31. The method of claim 29 or claim 30, wherein the anti-poly-GA antibody is a monoclonal antibody.

32. The method of any one of claims 28 to 31, wherein the inhibitory nucleic acid is double-stranded RNA (dsRNA), short-interfering RNA (siRNA), short-hairpin RNA (shRNA), microRNA (miRNA), artificial microRNA (amiRNA), an aptamer, or an antisense oligonucleotide (ASO).

33. The method of any one of claims 28 to 32, wherein the inhibitory nucleic acid comprises a region of complementarity with a nucleic acid sequence encoding a poly-GA repeat expansion in the subject.

34. The method of any one of claims 28 to 33, wherein the inhibitory nucleic acid comprises a region of complementarity with a nucleic acid sequence present at a chromosomal locus set forth in Table 2.

35. The method of any one of claims 28 to 34, wherein the inhibitory nucleic acid comprises a region of complementarity with an RNA transcript encoded by a gene, or a genetic locus thereof, selected from the group consisting of: ARMCX4, PEX14, PTPRF, ACTA1, DNAH14, PFN1P2, Clorf61, WASF2, PGBD2, NBPF15, DDX11L1, BARHL2, MIR1976, CASZ1, SLC44A3, GPR137B, SOX13, CROCC, RNPEP, MIR3121, MPZ, MCL1, HYDIN2, AIFM2, MGMT, LINC01164, KNDC1, ANK3, MLLT10, TBC1D12, LRMDA, CCNY, MIR3156-1, DUX4L2, AGAP12P, C10orf53, SMPD1, IFITM10, BUD13, TSPAN18, CD82, OTUB1, NADSYN1, MIR4492, CHID1, SMUG1, LINC00938, LINC01257, SLC15A4, ASIC1, DCP1B, TMTC2, TNS2, LOC100240735, SOX1, LATS2, RAB20, ANKRD20A9P, FLT1, RCBTB1, ELK2AP, STON2, FOXN3, TTLL5, BCL11B, BRMS1L, SMAD3, RPLP1, BAHD1, MYO5A, DNMIP46, DNMIP35, TUBGCP4, C16orf95, OSGIN1, LINC00311, MIR4718, RBFOX1, SBK1, MIR4722, BANP, C16orf78, MIR5189, ADGRG5, NPRL3, ZDHHC1, MIR662, LINC00482, MRPL12, TBC1D3H, WSCD1, TBC1D3B, TBC1D3, TBC1D3C, METRNL, DNAH9, ASGR1, FOXK2, NPEPPS, SARM1, CLUH, TIAF1, LOC440434, PHOSPHO1, TBCD, CYP4F35P, CXADRP3, LINC00668, MEX3C, COX7A1, SCAF1, RFPL4AL1, SIX5, DIRAS1, POLRMT, ZNF554, MED16, SIPAIL3, DOT1L, KMT5C, PDE4C, ZNF480, CEBPA, PTPN18, HAAO, LOC654342, RGPD2, RAB3GAP1, TNS1, FAM95A, NTSR1, FRG1BP, FAM182B, CDH4, PRNP, MIR1257, MIR4758, OGFR, SRC, COL9A3, ZNF512B, PICSAR, SIK1, CYP4F29P, MX1, LARGE1, CRELD2, UPK3A, RRP7A, MIR4762, SHANK3, SHISA8, CCDC188, NPTXR, ZNF621, TPRA1, PIGZ, LHFPL4, OSTN, GAP43, CACNA2D2, TNK2, IQSEC1, RAD18, PARP14, PLXNA1, DOCK3, DUX4L8, PCDH10, TNIP3, ZFYVE28, MSMO1, ANKRD50, FGFR4, IRX1, ZNF622, SPOCK1, PLEKHG4B, LCP2, SLC34A1, CXXC5, PPARGC1B, LOC643201, P4HA2, THBS2, SEC63, SLC17A5, MEA1, RIMS1, AR1D1B, PRKAG2, EN2, NXPH1, NUB1, DPP6, MYL10, GS1-124K5.11, ABCB4, MFSD3, SOX17, MTDH, RRS1-AS1, SDCBP, DOCKS, SHARPIN, LINC00051, LRRC6, NAPRT, FOXE1, C9orf139, FAM27C, AQP7P1, TLE4, NCS1, FAM27B, C9orf50, TOR1A, PNPLA7, MIR4473, PRRX2, DAB2IP, C9orf72, GPSM1, FAM230C, RNA5-8SN5, SUPT20HL2, SUPT20HL1, FAM236A, RPL10, AVPR2, SHROOM2, and FAM226A.

36. The method of any one of claims 28 to 35, wherein the administration of the one or more therapeutic agents to the subject results in a reduction of poly-GA RAN protein translation, expression, aggregation or accumulation in the subject, relative to the level of poly-GA RAN protein translation, expression, aggregation or accumulation in the subject prior to the administration.
